SEQUENCE LISTING (80 sequences)

SEQ ID NO: 1 - protein, 1922 aa, Homo sapiens
MKSPALQPLSMAGLQLMTPASSPMGPFFGLPWQQEAIHDNIYTPRKYQVELLEAALDHNTIVCLNTGSGKTFIAVLLTKELSYQIRGDFSRNGKRTVFLVNSANQVAQQVSAVRTHSDLKVGEYSNLEVNASWTKERWNQEFTKHQVLIMTCYVALNVLKNGYLSLSDINLLVFDECHLAILDHPYREIMKLCENCPSCPRILGLTASILNGKCDPEELEEKIQKLEKILKSNAETATDLVVLDRYTSQPCEIVVDCGPFTDRSGLYERLLMELEEALNFINDCNISVHSKERDSTLISKQILSDCRAVLVVLGPWCADKVAGMMVRELQKYIKHEQEELHRKFLLFTDTFLRKIHALCEEHFSPASLDLKFVTPKVIKLLEILRKYKPYERQQFESVEWYNNRNQDNYVSWSDSEDDDEDEEIEEKEKPETNFPSPFTNILCGIIFVERRYTAVVLNRLIKEAGKQDPELAYISSNFITGHGIGKNQPRNKQMEAEFRKQEEVLRKFRAHETNLLIATSIVEEGVDIPKCNLVVRFDLPTEYRSYVQSKGRARAPISNYIMLADTDKIKSFEEDLKTYKAIEKILRNKCSKSVDTGETDIDPVMDDDDVFPPYVLRPDDGGPRVTINTAIGHINRYCARLPSDPFTHLAPKCRTRELPDGTFYSTLYLPINSPLRASIVGPPMSCVRLAERVVALICCEKLHKIGELDDHLMPVGKETVKYEEELDLHDEEETSVPGRPGSTKRRQCYPKAIPECLRDSYPRPDQPCYLYVIGMVLTTPLPDELNFRRRKLYPPEDTTRCFGILTAKPIPQIPHFPVYTRSGEVTISIELKKSGFMLSLQMLELITRLHQYIFSHILRLEKPALEFKPTDADSAYCVLPLNVVNDSSTLDIDFKFMEDIEKSEARIGIPSTKYTKETPFVFKLEDYQDAVIIPRYRNFDQPHRFYVADVYTDLTPLSKFPSPEYETFAEYYKTKYNLDLTNLNQPLLDVDHTSSRLNLLTPRHLNQKGKALPLSSAEKRKAKWESLQNKQILVPELCAIHPIPASLWRKAVCLPSILYRLHCLLTAEELRAQTASDAGVGVRSLPADFRYPNLDFGWKKSIDSKSFISISNSSSAENDNYCKHSTIVPENAAHQGANRTSSLENHDQMSVNCRTLLSESPGKLHVEVSADLTAINGLSYNQNLANGSYDLANRDFCQGNQLNYYKQEIPVQPTTSYSIQNLYSYENQPQPSDECTLLSNKYLDGNANKSTSDGSPVMAVMPGTTDTIQVLKGRMDSEQSPSIGYSSRTLGPNPGLILQALTLSNASDGFNLERLEMLGDSFLKHAITTYLFCTYPDAHEGRLSYMRSKKVSNCNLYRLGKKKGLPSRMVVSIFDPPVNWLPPGYVVNQDKSNTDKWEKDEMTKDCMLANGKLDEDYEEEDEEEESLMWRAPKEEADYEDDFLEYDQEHIRFIDNMLMGSGAFVKKISLSPFSTTDSAYEWKMPKKSSLGSMPFSSDFEDFDYSSWDAMCYLDPSKAVEEDDFVVGFWNPSEENCGVDTGKQSISYDLHTEQCIADKSIADCVEALLGCYLTSCGERAAQLFLCSLGLKVLPVIKRTDREKALCPTRENFNSQQKNLSVSCAAASVASSRSSVLKDSEYGCLKIPPRCMFDHPDADKTLNHLISGFENFEKKINYRFKNKAYLLQAFTHASYHYNTITDCYQRLEFLGDAILDYLITKHLYEDPRQHSPGVLTDLRSALVNNTIFASLAVKYDYHKYFKAVSPELFHVIDDFVQFQLEKNEMQGMDSELRRSEEDEEKEEDIEVPKAMGDIFESLAGAIYMDSGMSLETVWQVYYPMMRPLIEKFSANVPRSPVRELLEMEPETAKFSPAERTYDGKVRVTVEVVGKGKFKGVGRSYRIAKSAAARRALRSLKANQPQVPNS

SEQ ID NO: 2 - DNA, 10323 bp, Homo sapiens
cggaggcgcggcgcaggctgctgcaggcccaggtgaatggagtaacctgacagcggggacgaggcgacggcgagcgcgaggaaatggcggcgggggcggcggcgccgggcggctccgggaggcctgggctgtgacgcgcgcgccggagcggggtccgatggttctcgaaggcccgcggcgccccgtgctgcagtaagctgtgctagaacaaaaatgcaatgaaagaaacactggatgaatgaaaagccctgctttgcaacccctcagcatggcaggcctgcagctcatgacccctgcttcctcaccaatgggtcctttctttggactgccatggcaacaagaagcaattcatgataacatttatacgccaagaaaatatcaggttgaactgcttgaagcagctctggatcataataccatcgtctgtttaaacactggctcagggaagacatttattgcagtactactcactaaagagctgtcctatcagatcaggggagacttcagcagaaatggaaaaaggacggtgttcttggtcaactctgcaaaccaggttgctcaacaagtgtcagctgtcagaactcattcagatctcaaggttggggaatactcaaacctagaagtaaatgcatcttggacaaaagagagatggaaccaagagtttactaagcaccaggttctcattatgacttgctatgtcgccttgaatgttttgaaaaatggttacttatcactgtcagacattaaccttttggtgtttgatgagtgtcatcttgcaatcctagaccacccctatcgagaaattatgaagctctgtgaaaattgtccatcatgtcctcgcattttgggactaactgcttccattttaaatgggaaatgtgatccagaggaattggaagaaaagattcagaaactagagaaaattcttaagagtaatgctgaaactgcaactgacctggtggtcttagacaggtatacttctcagccatgtgagattgtggtggattgtggaccatttactgacagaagtgggctttatgaaagactgctgatggaattagaagaagcacttaattttatcaatgattgtaatatatctgtacattcaaaagaaagagattctactttaatttcgaaacagatactatcagactgtcgtgccgtattggtagttctgggaccctggtgtgcagataaagtagctggaatgatggtaagagaactacagaaatacatcaaacatgagcaagaggagctgcacaggaaatttttattgtttacagacactttcctaaggaaaatacatgcactatgtgaagagcacttctcacctgcctcacttgacctgaaatttgtaactcctaaagtaatcaaactgctcgaaatcttacgcaaatataaaccatatgagcgacagcagtttgaaagcgttgagtggtataataatagaaatcaggataattatgtgtcatggagtgattctgaggatgatgatgaggatgaagaaattgaagaaaaagagaagccagagacaaattttccttctccttttaccaacattttgtgcggaattatttttgtggaaagaagatacacagcagttgtcttaaacagattgataaaggaagctggcaaacaagatccagagctggcttatatcagtagcaatttcataactggacatggcattgggaagaatcagcctcgcaacaaacagatggaagcagaattcagaaaacaggaagaggtacttaggaaatttcgagcacatgagaccaacctgcttattgcaacaagtattgtagaagagggtgttgatataccaaaatgcaacttggtggttcgttttgatttgcccacagaatatcgatcctatgttcaatctaaaggaagagcaagggcacccatctctaattatataatgttagcggatacagacaaaataaaaagttttgaagaagaccttaaaacctacaaagctattgaaaagatcttgagaaacaagtgttccaagtcggttgatactggtgagactgacattgatcctgtcatggatgatgatgacgttttcccaccatatgtgttgaggcctgacgatggtggtccacgagtcacaatcaacacggccattggacacatcaatagatactgtgctagattaccaagtgatccgtttactcatctagctcctaaatgcagaacccgagagttgcctgatggtacattttattcaactctttatctgccaattaactcacctcttcgagcctccattgttggtccaccaatgagctgtgtacgattggctgaaagagttgtagctctcatttgctgtgagaaactgcacaaaattggcgaactggatgaccatttgatgccagttgggaaagagactgttaaatatgaagaggagcttgatttgcatgatgaagaagagaccagtgttccaggaagaccaggttccacgaaacgaaggcagtgctacccaaaagcaattccagagtgtttgagggatagttatcccagacctgatcagccctgttacctgtatgtgataggaatggttttaactacacctttacctgatgaactcaactttagaaggcggaagctctatcctcctgaagataccacaagatgctttggaatactgacggccaaacccatacctcagattccacactttcctgtgtacacacgctctggagaggttaccatatccattgagttgaagaagtctggtttcatgttgtctctacaaatgcttgagttgattacaagacttcaccagtatatattctcacatattcttcggcttgaaaaacctgcactagaatttaaacctacagacgctgattcagcatactgtgttctacctcttaatgttgttaatgactccagcactttggatattgactttaaattcatggaagatattgagaagtctgaagctcgcataggcattcccagtacaaagtatacaaaagaaacaccctttgtttttaaattagaagattaccaagatgccgttatcattccaagatatcgcaattttgatcagcctcatcgattttatgtagctgatgtgtacactgatcttaccccactcagtaaatttccttcccctgagtatgaaacttttgcagaatattataaaacaaagtacaaccttgacctaaccaatctcaaccagccactgctggatgtggaccacacatcttcaagacttaatcttttgacacctcgacatttgaatcagaaggggaaagcgcttcctttaagcagtgctgagaagaggaaagccaaatgggaaagtctgcagaataaacagatactggttccagaactctgtgctatacatccaattccagcatcactgtggagaaaagctgtttgtctccccagcatactttatcgccttcactgccttttgactgcagaggagctaagagcccagactgccagcgatgctggcgtgggagtcagatcacttcctgcggattttagataccctaacttagacttcgggtggaaaaaatctattgacagcaaatctttcatctcaatttctaactcctcttcagctgaaaatgataattactgtaagcacagcacaattgtccctgaaaatgctgcacatcaaggtgctaatagaacctcctctctagaaaatcatgaccaaatgtctgtgaactgcagaacgttgctcagcgagtcccctggtaagctccacgttgaagtttcagcagatcttacagcaattaatggtctttcttacaatcaaaatctcgccaatggcagttatgatttagctaacagagacttttgccaaggaaatcagctaaattactacaagcaggaaatacccgtgcaaccaactacctcatattccattcagaatttatacagttacgagaaccagccccagcccagcgatgaatgtactctcctgagtaataaataccttgatggaaatgctaacaaatctacctcagatggaagtcctgtgatggccgtaatgcctggtacgacagacactattcaagtgctcaagggcaggatggattctgagcagagcccttctattgggtactcctcaaggactcttggccccaatcctggacttattcttcaggctttgactctgtcaaacgctagtgatggatttaacctggagcggcttgaaatgcttggcgactcctttttaaagcatgccatcaccacatatctattttgcacttaccctgatgcgcatgagggccgcctttcatatatgagaagcaaaaaggtcagcaactgtaatctgtatcgccttggaaaaaagaagggactacccagccgcatggtggtgtcaatatttgatccccctgtgaattggcttcctcctggttatgtagtaaatcaagacaaaagcaacacagataaatgggaaaaagatgaaatgacaaaagactgcatgctggcgaatggcaaactggatgaggattacgaggaggaggatgaggaggaggagagcctgatgtggagggctccgaaggaagaggctgactatgaagatgatttcctggagtatgatcaggaacatatcagatttatagataatatgttaatggggtcaggagcttttgtaaagaaaatctctctttctcctttttcaaccactgattctgcatatgaatggaaaatgcccaaaaaatcctccttaggtagtatgccattttcatcagattttgaggattttgactacagctcttgggatgcaatgtgctatctggatcctagcaaagctgttgaagaagatgactttgtggtggggttctggaatccatcagaagaaaactgtggtgttgacacgggaaagcagtccatttcttacgacttgcacactgagcagtgtattgctgacaaaagcatagcggactgtgtggaagccctgctgggctgctatttaaccagctgtggggagagggctgctcagcttttcctctgttcactggggctgaaggtgctcccggtaattaaaaggactgatcgggaaaaggccctgtgccctactcgggagaatttcaacagccaacaaaagaacctttcagtgagctgtgctgctgcttctgtggccagttcacgctcttctgtattgaaagactcggaatatggttgtttgaagattccaccaagatgtatgtttgatcatccagatgcagataaaacactgaatcaccttatatcggggtttgaaaattttgaaaagaaaatcaactacagattcaagaataaggcttaccttctccaggcttttacacatgcctcctaccactacaatactatcactgattgttaccagcgcttagaattcctgggagatgcgattttggactacctcataaccaagcacctttatgaagacccgcggcagcactccccgggggtcctgacagacctgcggtctgccctggtcaacaacaccatctttgcatcgctggctgtaaagtacgactaccacaagtacttcaaagctgtctctcctgagctcttccatgtcattgatgactttgtgcagtttcagcttgagaagaatgaaatgcaaggaatggattctgagcttaggagatctgaggaggatgaagagaaagaagaggatattgaagttccaaaggccatgggggatatttttgagtcgcttgctggtgccatttacatggatagtgggatgtcactggagacagtctggcaggtgtactatcccatgatgcggccactaatagaaaagttttctgcaaatgtaccccgttcccctgtgcgagaattgcttgaaatggaaccagaaactgccaaatttagcccggctgagagaacttacgacgggaaggtcagagtcactgtggaagtagtaggaaaggggaaatttaaaggtgttggtcgaagttacaggattgccaaatctgcagcagcaagaagagccctccgaagcctcaaagctaatcaacctcaggttcccaatagctgaaaccgctttttaaaattcaaaacaagaaacaaaacaaaaaaaattaaggggaaaattatttaaatcggaaaggaagacttaaagttgttagtgagtggaatgaattgaaggcagaatttaaagtttggttgataacaggatagataacagaataaaacatttaacatatgtataaaattttggaactaattgtagttttagttttttgcgcaaacacaatcttatcttctttcctcacttctgctttgtttaaatcacaagagtgctttaatgatgacatttagcaagtgctcaaaataattgacaggttttgtttttttttttttgagtttatgtcagctttgcttagtgttagaaggccatggagcttaaacctccagcagtccctaggatgatgtagattcttctccatctctccgtgtgtgcagtagtgccagtcctgcagtagttgataagctgaatagaaagataaggttttcgagaggagaagtgcgccaatgttgtcttttctttccacgttatactgtgtaaggtgatgttcccggtcgctgttgcacctgatagtaagggacagatttttaatgaacattggctggcatgttggtgaatcacattttagttttctgatgccacatagtcttgcataaaaaagggttcttgccttaaaagtgaaaccttcatggatagtctttaatctctgatctttttggaacaaactgttttacattcctttcattttattatgcattagacgttgagacagcgtgatacttacaactcactagtatagttgtaacttattacaggatcatactaaaatttctgtcatatgtatactgaagacattttaaaaaccagaatatgtagtctacggatattttttatcataaaaatgatctttggctaaacaccccattttactaaagtcctcctgccaggtagttcccactgatggaaatgtttatggcaaataattttgccttctaggctgttgctctaacaaaataaaccttagacatatcacacctaaaatatgctgcagattttataattgattggttacttatttaagaagcaaaacacagcacctttacccttagtctcctcacataaatttcttactatacttttcataatgttgcatgcatatttcacctaccaaagctgtgctgttaatgccgtgaaagtttaacgtttgcgataaactgccgtaattttgatacatctgtgatttaggtcattaatttagataaactagctcattatttccatctttggaaaaggaaaaaaaaaaaaacttctttaggcatttgcctaagtttctttaattagacttgtaggcactcttcacttaaatacctcagttcttcttttcttttgcatgcatttttcccctgtttggtgctatgtttatgtattatgcttgaaattttaatttttttttttttgcactgtaactataatacctcttaatttacctttttaaaagctgtgggtcagtcttgcactcccatcaacataccagtagaggtttgctgcaatttgccccgttaattatgcttgaagtttaagaaagctgagcagaggtgtctcatatttcccagcacatgattctgaacttgatgcttcgtggaatgctgcatttatatgtaagtgacatttgaatactgtccttcctgctttatctgcatcatccacccacagagaaatgcctctgtgcgagtgcaccgacagaaaactgtcagctctgctttctaaggaaccctgagtgaggggggtattaagcttctccagtgttttttgttgtctccaatcttaaacttaaattgagatctaaattattaaacgagtttttgagcaaattaggtgacttgttttaaaaatatttaattccgatttggaaccttagatgtctatttgattttttaaaaaaccttaatgtaagatatgaccagttaaaacaaagcaattcttgaattatataactgtaaaagtgtgcagttaacaaggctggatgtgaattttattctgagggtgatttgtgatcaagtttaatcacaaatctcttaatatttataaactacctgatgccaggagcttagggctttgcattgtgtctaatacattgatcccagtgttacgggattctcttgattcctggcaccaaaatcagattgttttcacagttatgattcccagtgggagaaaaatgcctcaatatatttgtaaccttaagaagagtatttttttgttaatactaagatgttcaaacttagacatgattaggtcatacattctcaggggttcaaatttccttctaccattcaaatgttttatcaacagcaaacttcagccgtttcactttttgttggagaaaaatagtagattttaatttgactcacagtttgaagcattctgtgatcccctggttactgagttaaaaaataaaaaagtacgagttagacatatgaaatggttatgaacgcttttgtgctgctgatttttaatgctgtaaagttttcctgtgtttagcttgttgaaatgttttgcatctgtcaattaaggaaaaaaaaaatcactctatgttgccccactttagagccctgtgtgccaccctgtgttcctgtgattgcaatgtgagaccgaatgtaatatggaaaacctaccagtggggtgtggttgtgccctgagcacgtgtgtaaaggactggggaggcgtgtcttgaaaaagcaactgcagaaattccttatgatgattgtgtgcaagttagttaacatgaaccttcatttgtaaattttttaaaatttcttttataatatgctttccgcagtcctaactatgctgcgttttataatagctttttcccttctgttctgttcatgtagcacagataagcattgcacttggtaccatgctttacctcatttcaagaaaatatgcttaacagagaggaaaaaaatgtggtttggccttgctgctgttttgatttatggaatttgaaaaagataattataatgcctgcaatgtgtcatatactcgcacaacttaaataggtcatttttgtctgtggcatttttactgtttgtgaaagtatgaaacagatttgttaactgaactcttaattatgtttttaaaatgtttgttatatttcttttcttttttcttttatattacgtgaagtgatgaaatttagaatgacctctaacactcctgtaattgtcttttaaaatactgatatttttatttgttaataatactttgccctcagaaagattctgataccctgccttgacaacatgaaacttgaggctgctttggttcatgaatccaggtgttcccccggcagtcggcttcttcagtcgctccctggaggcaggtgggcactgcagaggatcactggaatccagatcgagcgcagttcatgcacaaggccccgttgatttaaaatattggatcttgctctgttagggtgtctaatccctttacacaagattgaagccaccaaactgagaccttgatacctttttttaactgcatctgaaattatgttaagagtctttaacccatttgcattatctgcagaagagaaactcatgtcatgtttattacctatatggttgttttaattacatttgaataattatatttttccaaccactgattacttttcaggaatttaattatttccagataaatttctttattttatattgtacatgaaaagttttaaagatatgtttaagaccaagactattaaaatgatttttaaagttgttggagacgccaatagcaatatctaggaaatttgcattgagaccattgtattttccactagcagtgaaaatgatttttcacaactaacttgtaaatatattttaatcattacttctttttttctagtccatttttatttggacatcaaccacagacaatttaaattttatagatgcactaagaattcactgcagcagcaggttacatagcaaaaatgcaaaggtgaacaggaagtaaatttctggcttttctgctgtaaatagtgaaggaaaattactaaaatcaagtaaaactaatgcatattatttgattgacaataaaatatttaccatcacatgctgcagctgttttttaaggaacatgatgtcattcattcatacagtaatcatgctgcagaaatttgcagtctgcaccttatggatcacaattacctttagttgttttttttgtaataattgtagccaagtaaatctccaataaagttatcgtctgttcaaaaaaaaaaaaaaaaaaaaaaaaaaaaaaaaaaaaaaaaaaaaaaa

SEQ ID NO: 3 - protein, 1922 aa, Homo sapiens; identical to SEQ ID NO: 1

SEQ ID NO: 4 - DNA, 10323 bp, Homo sapiens; identical to SEQ ID NO: 2

SEQ ID NO: 5 - protein, 1922 aa, Homo sapiens; identical to SEQ ID NO: 1

SEQ ID NO: 6 - DNA, 37 bp, Homo sapiens
cgattttatgtagctgatgtgtacactgatcttaccc

SEQ ID NO: 7 - DNA, 29 bp, Homo sapiens
aaggcggaagctctatcctcctgaagata

SEQ ID NO: 8 - DNA, 39 bp, Homo sapiens
tctgttcactggggctgaaggtgctcccggtaattaaaa

SEQ ID NO: 9 - DNA, 31 bp, Homo sapiens
cagatggaagcagaattcagaaaacaggaag

SEQ ID NO: 10 - DNA, 32 bp, Homo sapiens
actgtgctagattaccaagtgatccgtttact

SEQ ID NO: 11 - DNA, 28 bp, Homo sapiens
atgttagcggatacagacaaaataaaaa

SEQ ID NO: 12 - DNA, 27 bp, Homo sapiens
gttccacgaaacgaaggcagtgctacc

SEQ ID NO: 13 - DNA, 29 bp, Homo sapiens
atcttacagcaattaatggtctttcttac

SEQ ID NO: 14 - DNA, 27 bp, Homo sapiens
ttcgttttgatttgcccacagaatatc

SEQ ID NO: 15 - DNA, 36 bp, Homo sapiens
ggaagaccaggttccacgaaacgaaggcagtgctac

SEQ ID NO: 16 - DNA, 21 bp, Artificial Sequence
  Primers
tcaaatccaattacccagcag

SEQ ID NO: 17 - DNA, 23 bp, Artificial Sequence
  Primers
tctgccagaagagattaaatgag

SEQ ID NO: 18 - DNA, 23 bp, Artificial Sequence
  Primers
aaatcagacaaccaaggctacag

SEQ ID NO: 19 - DNA, 27 bp, Artificial Sequence
  Primers
tttaatattcattcattcatacactgc

SEQ ID NO: 20 - DNA, 23 bp, Artificial Sequence
  Primers
gaattcttactcttgcccattcc

SEQ ID NO: 21 - DNA, 22 bp, Artificial Sequence
  Primers
gagccgcattaagcatattttc

SEQ ID NO: 22 - DNA, 20 bp, Artificial Sequence
  Primers
tcacatcacaacacaggacg

SEQ ID NO: 23 - DNA, 25 bp, Artificial Sequence
  Primers
aaatcactctacagctacctcatgg

SEQ ID NO: 24 - DNA, 27 bp, Artificial Sequence
  Primers
ttcctatggatacaaagaataacaaag

SEQ ID NO: 25 - DNA, 24 bp, Artificial Sequence
  Primers
aacttttattgctgcacgatactg

SEQ ID NO: 26 - DNA, 25 bp, Artificial Sequence
  Primers
tgaacatgtagatgactacaaaagc

SEQ ID NO: 27 - DNA, 22 bp, Artificial Sequence
  Primers
aagtgttcatggtgcatgattc

SEQ ID NO: 28 - DNA, 20 bp, Artificial Sequence
  Primers
aagctgtgaatcggagaaag

SEQ ID NO: 29 - DNA, 25 bp, Artificial Sequence
  Primers
tctagtggagaaatagaagaggcac

SEQ ID NO: 30 - DNA, 24 bp, Artificial Sequence
  Primers
ttttagtagagacgaggtttcacc

SEQ ID NO: 31 - DNA, 20 bp, Artificial Sequence
  Primers
tttgtgtgcaaagcatctcc

SEQ ID NO: 32 - DNA, 24 bp, Artificial Sequence
  Primers
tttgtgatatattaatgggccaag

SEQ ID NO: 33 - DNA, 24 bp, Artificial Sequence
  Primers
tctcactccaactgttatggctta

SEQ ID NO: 34 - DNA, 20 bp, Artificial Sequence
  Primers
gagtacattcatcgctgggc

SEQ ID NO: 35 - DNA, 20 bp, Artificial Sequence
  Primers
actgcaaaccactttcaggc

SEQ ID NO: 36 - DNA, 20 bp, Artificial Sequence
  Primers
agaaatttgcctccatcaaa

SEQ ID NO: 37 - DNA, 19 bp, Artificial Sequence
  Primers
cagggcttccacacagtcc

SEQ ID NO: 38 - DNA, 20 bp, Artificial Sequence
  Primers
tacaaggccaacacgatgag

SEQ ID NO: 39 - DNA, 20 bp, Artificial Sequence
  Primers
tgccgtcagaactctgaaac

SEQ ID NO: 40 - DNA, 20 bp, Artificial Sequence
  Primers
tgaacttttcccctttgatg

SEQ ID NO: 41 - DNA, 20 bp, Artificial Sequence
  Primers
tctgccttcaattcattcca

SEQ ID NO: 42 - DNA, 22 bp, Artificial Sequence
  Primers
gcaatgaaagaaacactggatg

SEQ ID NO: 43 - DNA, 23 bp, Artificial Sequence
  Primers
ttttgtaaatttattggaggacg

SEQ ID NO: 44 - DNA, 22 bp, Artificial Sequence
  Primers
ttttggaggataaccttggaac

SEQ ID NO: 45 - DNA, 21 bp, Artificial Sequence
  Primers
ttgtcgtcaagacatgctttc

SEQ ID NO: 46 - DNA, 20 bp, Artificial Sequence
  Primers
tagtggcatttccaccaaac

SEQ ID NO: 47 - DNA, 20 bp, Artificial Sequence
  Primers
cccactgctaacattctggc

SEQ ID NO: 48 - DNA, 20 bp, Artificial Sequence
  Primers
aaatcccagttaaaccccac

SEQ ID NO: 49 - DNA, 20 bp, Artificial Sequence
  Primers
taaatcaccgtcgccaaatc

SEQ ID NO: 50 - DNA, 24 bp, Artificial Sequence
  Primers
catgtgtgtcagaaatgacagttg

SEQ ID NO: 51 - DNA, 25 bp, Artificial Sequence
  Primers
agcaggttactttggagtactgaag

SEQ ID NO: 52 - DNA, 20 bp, Artificial Sequence
  Primers
tcacatttcaagtgctcacc

SEQ ID NO: 53 - DNA, 27 bp, Artificial Sequence
  Primers
ttttactaggcaggacttttaaagatg

SEQ ID NO: 54 - DNA, 21 bp, Artificial Sequence
  Primers
tttgcagtccagctcatattg

SEQ ID NO: 55 - DNA, 20 bp, Artificial Sequence
  Primers
taagaagtgtcatgcctcgg

SEQ ID NO: 56 - DNA, 24 bp, Artificial Sequence
  Primers
gaaagcatcatttctgttctgaag

SEQ ID NO: 57 - DNA, 22 bp, Artificial Sequence
  Primers
tgtaaaggtgccatttagcttc

SEQ ID NO: 58 - DNA, 23 bp, Artificial Sequence
  Primers
attgcacttgagggattcttacc

SEQ ID NO: 59 - DNA, 21 bp, Artificial Sequence
  Primers
ttggcccattaatatatcaca

SEQ ID NO: 60 - DNA, 20 bp, Artificial Sequence
  Primers
aattgctgttgctctcagcc

SEQ ID NO: 61 - DNA, 20 bp, Artificial Sequence
  Primers
acaagcaggaaatacccgtg

SEQ ID NO: 62 - DNA, 23 bp, Artificial Sequence
  Primers
aaagcatagaatatgtgggaatt

SEQ ID NO: 63 - DNA, 23 bp, Artificial Sequence
  Primers
aacccttgcttttattgagtttc

SEQ ID NO: 64 - DNA, 20 bp, Artificial Sequence
  Primers
aaactgtggtgttgacacgg

SEQ ID NO: 65 - DNA, 23 bp, Artificial Sequence
  Primers
tgtggggatagtgtaaatgcttc

SEQ ID NO: 66 - DNA, 20 bp, Artificial Sequence
  Primers
tggactgcctgtaaaagtgg

SEQ ID NO: 67 - DNA, 19 bp, Artificial Sequence
  Primers
cctgtctgtcgggggtatg

SEQ ID NO: 68 - DNA, 20 bp, Artificial Sequence
  Primers
aatcacaggctcgctctcat

SEQ ID NO: 69 - DNA, 18 bp, Artificial Sequence
  Primers
gtctccacctccgctgct

SEQ ID NO: 70 - DNA, 23 bp, Artificial Sequence
  Primers
gggaaagcagtccatttcttacg

SEQ ID NO: 71 - DNA, 20 bp, Artificial Sequence
  Primers
accttcagccccagtgaaca

SEQ ID NO: 72 - DNA, 15 bp, Artificial Sequence
  Primers
tcagccccagtgaac

SEQ ID NO: 73 - DNA, 20 bp, Artificial Sequence
  Primers
cctgatcagccctgttacct

SEQ ID NO: 74 - DNA, 26 bp, Artificial Sequence
  Primers
tgtggaaagaagatacacagcagttg

SEQ ID NO: 75 - DNA, 21 bp, Artificial Sequence
  Primers
cacctcttcgagcctccattg

SEQ ID NO: 76 - DNA, 21 bp, Artificial Sequence
  Primers
cacctcttcgagcctccattg

SEQ ID NO: 77 - DNA, 20 bp, Artificial Sequence
  Primers
cctgatcagccctgttacct

SEQ ID NO: 78 - DNA, 20 bp, Artificial Sequence
  Primers
ttggtctcatgtgctcgaaa

SEQ ID NO: 79 - DNA, 21 bp, Artificial Sequence
  Primers
gggctgatcaggtctgggata

SEQ ID NO: 80 - DNA, 21 bp, Artificial Sequence
  Primers
gggctgatcaggtctgggata

(19) United States
(12) Patent Application Publication
(10) Pub. No.: US 2012/0040357 A1
(43) Pub. Date: Feb. 16, 2012

(54) COMPOSITIONS, METHODS AND KITS TO DETECT DICER GENE MUTATIONS

(75) Inventors: Ashley D. Hill, Arlington, VA (US); Paul Goodfellow, St. Louis, MO (US); John R. Priest, Minneapolis, MN (US); Yoav Messinger, Minneapolis, MN (US)

(73) Assignees: THE WASHINGTON UNIVERSITY IN SAINT LOUIS, St. Louis, MO (US); CHILDREN'S HOSPITAL AND CLINICS OF MINNESOTA, Minneapolis, MN (US)

(21) Appl. No.: 13/139,671

(22) PCT Filed: Dec. 18, 2009
(86) PCT No.: PCT/US2009/068691

§ 371 Date: Oct. 26, 2011

Related U.S. Application Data
(60) Provisional application No. 61/138,875, filed on Dec. 18, 2008.
(60) Provisional application No. 61/169,474, filed on Apr. 15, 2009.

(51) Int. Cl. C12Q 1/68; C07H 21/04

(52) U.S. Cl. 435/6.12; 536/23.2; 536/24.33; 536/24.3

(57) ABSTRACT

In one aspect, the disclosure provides isolated nucleic acids, primers, and probes for the detection of mutations in a nucleic acid sequence for a DICER1 polypeptide.

This application is being filed on 18 Dec. 2009, as a PCT International Patent application in the name of Children's Hospital and Clinics of Minnesota, a U.S. national corporation, and The Washington University in Saint Louis, a corporation established by the special act of the Missouri General Assembly, applicants for the designation of all countries except the U.S., and D. Ashley Hill, a citizen of the U.S., Paul Goodfellow, a citizen of the U.S., John R. Priest, a citizen of the U.S., and Yoav Messinger, a citizen of the U.S., applicants for the designation of the U.S. only, and claims priority to U.S. Provisional Patent Application Ser. No. 61/138,875 filed on 18 Dec. 2008 and U.S. Provisional Patent Application Ser. No. 61/169,474 filed on 15 Apr. 2009.

### BACKGROUND OF THE INVENTION

Pleuropulmonary blastoma (PPB) is a rare childhood sarcoma of the lung that is thought to arise in fetal and infant lung development. As a lung cancer, PPB is similar to more common cancers of other tissues in children (such as kidney, liver, or muscle). These cancers look embryonic under the microscope and appear to be disorders of organ growth occurring in this phase of childhood. These malignancies include nephroblastoma (Wilms tumor), neuroblastoma, hepatoblastoma and embryonal rhabdomyosarcoma.

PPB often begins as a cyst in the lung. These cysts appear to be congenital malformations of the lung but have very subtle signs of malignancy. Over two to four years, these early malignant cysts develop into full-blown aggressive solid tumors of the lung. Three clinically distinct but related forms of PPB are recognized. Type I PPB, the early stage of tumor development, is characterized by formation of cysts in the lung parenchyma. These cysts are lined by normal-appearing alveolar or bronchiolar-type epithelium and appear to represent expanded alveolar spaces that lack typical septal branching pattern (Hill et al. Am. J. Surg. Pathol. 32 (2008): 282-95). Mesenchymal cells susceptible to malignant transformation reside within the cyst walls and have the potential to differentiate along multiple lineages, especially skeletal muscle and cartilage. Type II and type III PPB represent later stages of tumorigenesis with progressive overgrowth of cysts by a multi-patterned sarcoma with accompanying anaplasia. The mesenchymal cells in the cyst wall proliferate forming cystic and solid tumors in type II PPB or purely solid tumors in type III PPB. Early diagnosis is imperative to decreasing the morbidity and mortality of disease.

PPB has a strong genetic susceptibility. Approximately 20% of children with PPB have additional lung cysts or lung and kidney cysts. In addition, the PPB patient or close family members have diseases such as PPB, lung cysts, kidney cysts or sarcomas. (Boman et al. J. Pediatr. 149:850 (2006). Analysis of genetic alterations in patients with the malignant PPB can be useful to identify genetic markers that adversely impact developmentally-timed programs in lung branching morphogenesis and also confer risk for malignant transformation.

### SUMMARY

In one aspect, the disclosure provides isolated nucleic acids, primers, and probes for the detection of mutations in a nucleic acid sequence for a DICER1 polypeptide. In embodiments, the disclosure provides an isolated nucleic acid that comprises a portion of a genomic sequence for DICER1, wherein the portion of the genomic sequence comprises a nucleotide position that can be mutated as compared to a reference sequence (such as SEQ ID NO:2), wherein when the nucleotide position is mutated a function of DICER1 is decreased or altered. In embodiments, the isolated nucleic acid sequence is less than a full length cDNA or genomic sequence, and/or less than a genomic exon sequence. In embodiments, the isolated nucleic acid sequence can have about 80 to 100%, including each percentage in between these numbers, sequence identity to a reference sequence such as SEQ. ID NO:2.

In other embodiments, an isolated nucleic acid specifically hybridizes or binds to the isolated nucleic acid that comprises a portion of the nucleic acid sequence for DICER1, wherein the nucleic acid preferentially hybridizes to the sequence comprising the mutation at the nucleotide position as compared to a sequence lacking the mutation is provided. In a specific embodiment, the isolated nucleic acid only binds to the sequence with the mutation. In other embodiments, an isolated nucleic acid specifically hybridizes to the genomic sequence of claim 1, wherein the nucleic acid preferentially hybridizes to the sequence without the mutation at the nucleotide position as compared to a sequence with the mutation at that location such as the wild type or reference sequence. In a specific embodiment, the isolated nucleic acid only binds to the wild type or reference sequence.

Another aspect of the disclosure includes methods and kits for diagnosis, prognosis, and treatment for cancer. In some embodiments, a sample from a subject can be screened for the presence of one or more DICER1 mutations. The presence of a DICER1 mutation is indicative of an increased risk that cancer will develop in the subject or the children of the subject. In some embodiments, the DICER 1 mutation detected is one that results in a loss of one or more functions of DICER 1. The samples can include cells or tissue from, without limitation, germ cells, embryos, biopsy tissue, blood samples, lung tissue, and kidney tissue. In some embodiments, the cancers are selected from the group consisting of PBB, cystic nephroma, renal cysts, thyroid carcinoma, thyroid nodular hyper plasias, bladder rhabdomyosarcoma, intestinal polyps, leukemia, ovarian germ cell tumors, testicular germ cell tumors, ovarian dysgerminoma, testicular seminoma, hepatic hamartomas, nasal chondromesenchymal hamartoma, Wilms tumor, rhabdomyosarcoma, synovial sarcoma, Sertoli-Leydig tumors, medulloblastoma, glioblastoma multiforme, primary brain sarcoma, ependymoma, neuroblastoma, and neurofibromatosis Type I. In embodiments, the method comprises determining whether the nucleic acid encoding DICER1 or the genomic sequence of DICER1 has the reference sequence or a mutated sequence, wherein the presence of the mutated sequence is indicative of a change in DICER1 such as a loss of function and/or alteration in structure and/or the presence of cancer.

In other embodiments, the cancer has a mesenchymal and epithelial component, and a sample may include one or both cell types. Other cancers that have an epithelial and mesenchymal component include carcinosarcoma and/or sarcomatoid cancers of the breast, uterus, lung, and gastrointestinal tract, malignant mesothelioma, sex chord stromal tumors, and ameloblastoma. In some embodiments, the cancer can also be characterized by having an epithelial to mesenchymal transition by identifying a change in other markers such as e-cadherins and/or based on histopathology of a tumor sample. Such transitions are also associated with an increased risk of metastasis.

Detection of the presence or absence of at least one mutation in nucleic acid sequence encoding or a genomic sequence of DICER1 can be determined using many different methods known to those of skill in the art. In some embodiments, a genomic sequence is analyzed for one or more of the mutations as shown in Table 1. Probes and/or primers are designed to detect the presence or absence of a mutation in the nucleic acid sequence. Alternatively, altered DICER1 polypeptide can be detected, including but not limited to truncated polypeptides, polypeptides with altered sequences, or polypeptides with a loss of one or more functions of DICER1.

In other embodiments other mutations that result in a loss of DICER 1 function may be detected. Such mutations may include those that result in a truncation or frameshift such that the RNase domains of DICER1 are not functional. The genomic sequence or a portion thereof can be isolated and sequenced. In other embodiments, all or a portion of the genomic sequence can be contacted with a probe that specifically hybridizes to the wild type sequence at the location of a mutation and any mismatch between the probe and the genomic sequence can be detected either chemically, or enzymatically. In other embodiments, probes specific for either wild type or mutated sequence can be used to determine which sequence is present in a sample. In some embodiments, primers are designed that can amplify mRNA or genomic DNA. In some embodiments, the primers are those that are shown in Tables 2A, 2B, and 2C. Amplified products can be sequenced to identify whether a mutation is present or the amplified products can be contacted with a probe that specifically binds to a sequence that is the wild type and a probe that specifically binds to a sequence that contains the mutation.

In another aspect of the disclosure, a method of treating cancer is provided comprising administering a nucleic acid encoding a DICER 1 polypeptide or a DICER 1 polypeptide to a tumor cell or surrounding tissue, wherein the DICER1 polypeptide has RNAse activity.

### BRIEF DESCRIPTION OF THE DRAWINGS

FIG. 1. Mapping the PPB susceptibility locus on distal 14q and identification of DICER1 mutations. Pedigrees for the four families included in the linkage analysis. A) Probands are indicated by arrows. Individuals with PPB, PPB-related lung cysts, cystic nephroma or embryonal rhabdomyosarcoma (ERMS) are shown as filled in symbols. Circles represent females, squares represent males. Symbols with a slash through them indicate deceased individuals. Generations are listed Ito IV and individual family members are identified by number. Individuals genotyped for linkage analysis are indicated with an asterisk. For individual IV-1 (#) from Family L genotypes were determined by RFLP analysis using DNA prepared from FFPE tissue. B) Genome-wide linkage analysis yielded a peak parametric LOD score of 3.71 at 14q31.1-32 for the four families. This analysis included 3736 markers and classified obligate carriers with normal phenotypes as “unaffected.”

FIG. 2 DICER1 mutations in PPB A. Unique DICER1 sequence alterations present in the probands of each of the four families. B. Location of mutations in DICER1 protein in 10 PPB families. Four-point stars represent truncating mutations and the arrow marks the location of the missense mutation.

FIG. 3. DICER1 staining in normal and tumor-associated epithelium. (A) Cytoplasmic DICER1 protein staining is seen in both epithelial and mesenchymal components in this 13 week gestation fetal lung. (B) Cytoplasmic DICER1 protein staining of normal lung in 18 month-old child from Family X whose tumor epithelium is shown below in (D). (C to E) Six of seven PPBs with an epithelial component to the tumor showed absent staining in the surface epithelial cells (arrows) but retention of staining of the mesenchymal tumor cells (representative fields from three separate tumors from Families C, D, E shown here). Note Family C had a missense mutation but still lacks DICER1 protein expression by immunohistochemistry. (F) One of the seven tumors with epithelial component showed positive staining in the epithelium in the single slide available for analysis (Family G). [Rabbit polyclonal anti-DICER1 with hematoxylin counterstain. Original magnifications ×200 (A); ×400 (B-F).]

FIG. 4: Reduction in mutant mRNA and absence of truncated protein in lymphoblasts from mutation carriers. (A) Sequence analysis of RT-PCR products (mRNA) from an affected member of family L in which the A substitution mutation (arrow) is much reduced compared to the genomic DNA (gDNA) in which wild-type C and mutant A peak heights are essentially equal (arrow). (B) Sequence of RT-PCR products from an affected member of family G with overlapping sequences attributable to the TACC insertion mutation (mRNA) in which the wild-type sequences predominate. Sequencing RT-PCR conformational variants (nondenaturing acrylamide gel separation) confirmed the presence of both mutant (conformer 1) and wild-type (conformer 2) transcripts. (C) Western blot analysis detection of only the full length ˜218 kDa DICER1 protein (arrowhead) in lymphoblasts from PPB mutation carriers. The mutation in family B leads to a DICER1 truncation that would result in a protein with a predicted size of 98.7 kDa. Family L has a truncation N-terminal to the epitope recognized by the 13D6 antibody. The ˜218 kDa protein (arrow) and the same non-specific bands are seen in lymphoblasts from PPB patients and the MFE and AN3CA control (endometrial cancer) cell lines. Marker (M) sizes in kDa are indicated.

### DETAILED DESCRIPTION

Unless defined otherwise, all technical and scientific terms used herein have the same meaning as commonly understood by one of ordinary skill in the art to.

### DEFINITIONS

An “allele” refers to any of two or more alternative forms of a gene that occupy the same locus on a chromosome. If two alleles within a diploid individual are identical by descent (that is, both alleles are direct descendants of a single allele in an ancestor), such alleles are called autozygous. If the alleles are not identical by descent, they are called allozygous. If two copies of same allele are present in an individual, the individual is homozygous for that allelic form of the gene. If different alleles are present in an individual, the individual is heterozygous for that gene.

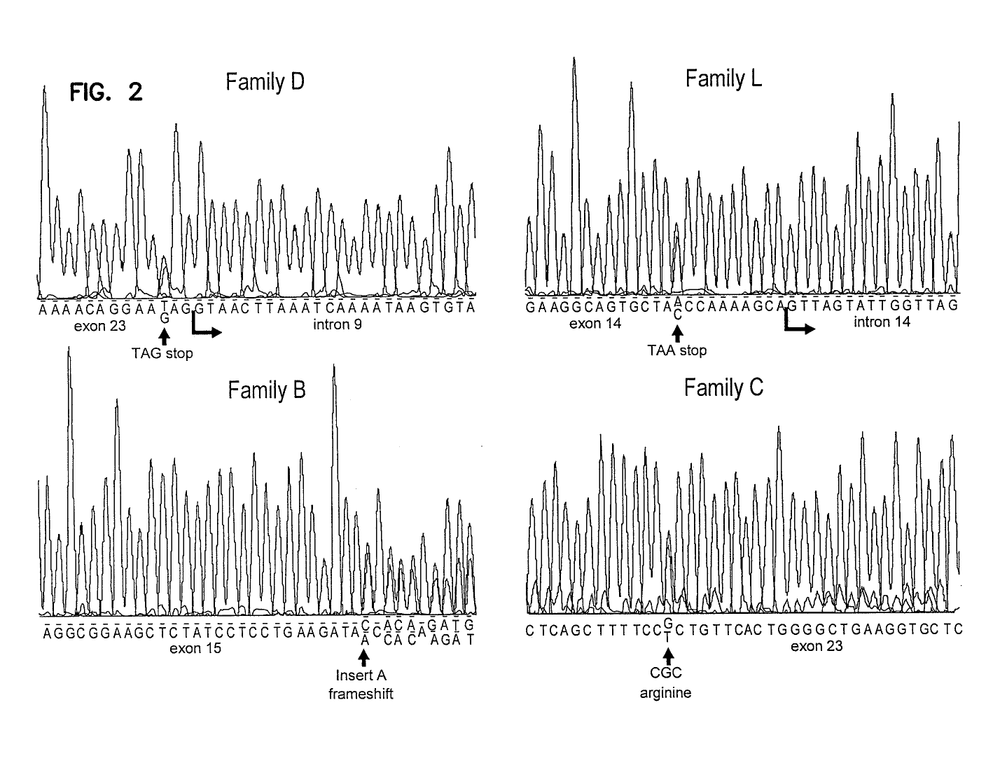

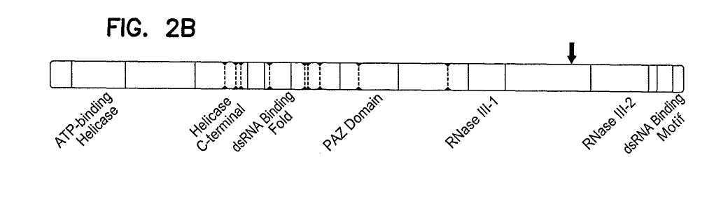

Unless otherwise expressly provided, the term “DICER1”, is used herein to refer to all species of nucleic acids encoding DICER 1 polypeptides, including all transcript variants. Reference sequences for DICER1 can be obtained from publicly available databases. A nucleic acid reference sequence for DICER1 has Gen Bank accession no. NM_{—}177438; GI 168693430(build 36.1) (Table 4; SEQ ID NO:2) and can be used as a reference sequence for assembly and primer construction. A polypeptide reference sequence for a DICER1 polypeptide has Gen Bank accession no. NP_{—}803187; GI 29294651(Table 3, SEQ ID NO:1). The amino acid numbering used begins with the Kozak sequence. DICER 1 genomic sequence contains 27 exons and various domains as shown in FIG. 2C including ATP binding helicase domain, Helicase C terminal domain, ds RNAbinding fold domain, PAZ domain, RNAse II-1 and III-2 domains, and ds RNA binding motif. The locations of the exons, the location and sequences of the introns, and the location of the domains have been described.

“Locked Nucleic Acids” or “LNA” as used herein refer to a class of nucleic acid analogues in which the ribose ring is “locked” by a methylene bridge connecting the 2′-O atom with the 4′-C atom. LNA nucleosides contain the six common nucleobases (T, C, G, A, U and mC) that appear in DNA and RNA and thus are able to form base-pairs according to standard Watson-Crick base pairing rules. Oligonucleotides incorporating LNA have increased thermal stability and improved discriminative power with respect to their nucleic acid targets. LNA can be mixed with DNA, RNA and other nucleic acid analogs using standard phosphoramidite synthesis chemistry. LNA oligonucleotides can easily be labeled with standard oligonucleotide tags such as DIG, fluorescent dyes, biotin, amino-linkers, etc.

“Molecular beacons” or “MB” as used herein refer to a probe comprising a fluorescent label attached to one end of a polynucleotide and a quencher attached to the other. Complementary base-pairs near the label and quencher cause a hairpin-like structure, placing the fluorophore and quencher in proximity. This hairpin opens in the presence of the target producing an increase in fluorescence. The proximity of the quencher to the fluorophore can result in reductions of fluorescent intensity of up to 98%. The efficiency can further be adjusted by altering the stem strength (length of the stem) which affects the number of beacons in the open state in the absence of the target.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic nucleic acid adaptors or linkers are used in accordance with conventional practice.

“Percent (%) amino acid sequence identity” with respect to the polypeptide sequences referred to herein is defined as the percentage of amino acid residues in a candidate sequence that are identical with the amino acid residues in a sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, or Megalign (DNASTAR) software. Those skilled in the art can determine appropriate parameters for measuring alignment, including any algorithms needed to achieve maximal alignment over the full-length of the sequences being compared.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Amino acid sequence identity may be determined using the sequence comparison program NCBI-BLAST2 (Altschul et al., Nucleic Acids Res. 25:3389-3402 (1997)). The NCBI-BLAST2 sequence comparison program may be downloaded from ncbi.nlm.nih.gov. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

For purposes herein, the % nucleic acid sequence identity of a given nucleic acid sequence A to, with, or against a given nucleic acid sequence B (which can alternatively be phrased as a given nucleic acid sequence A that has or comprises a certain % nucleic acid sequence identity to, with, or against a given amino acid sequence B) is calculated as follows:

100 times the fraction X/Y

where X is the number of nucleic acid residues scored as identical matches by the sequence alignment program's alignment of A and B, and where Y is the total number of nucleic acid residues in B. It will be appreciated that where the length of nucleic acid sequence A is not equal to the length of nucleic acid sequence B, the % nucleic acid sequence identity of A to B will not equal the % nucleic acid sequence identity of B to A. Nucleic acid sequence identity may be determined using the sequence comparison program NCBI-BLAST2 (Altschul et al., Nucleic Acids Res. 25:3389-3402 (1997)). The NCBI-BLAST2 sequence comparison program may be downloaded from ncbi.nlm.nih.gov. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for nucleic acid sequence comparisons, the % nucleic acid sequence identity of a given nucleic acid sequence A to, with, or against a given nucleic acid sequence B (which can alternatively be phrased as a given nucleic acid sequence A that has or comprises a certain % nucleic acid sequence identity to, with, or against a given nucleic acid sequence B) is calculated as follows:

100 times the fraction X/Y

where X is the number of nucleic acid residues scored as identical matches by the sequence alignment program NCBI-BLAST2 in that program's alignment of A and B, and where Y is the total number of nucleic acid residues in B. It will be appreciated that where the length of nucleic acid sequence A is not equal to the length of nucleic acid sequence B, the % nucleic acid sequence identity of A to B will not equal the % nucleic acid sequence identity of B to A.

“Polymerase chain reaction” or “PCR” refers to a procedure or technique in which minute amounts of a specific piece of nucleic acid, RNA and/or DNA, are amplified as described in U.S. Pat. No. 4,683,195 issued Jul. 28, 1987. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers can coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al., Cold Spring Harbor Symp. Quant. Biol. 51:263 (1987); Erlich, ed., PCR Technology (Stockton Press, NY, 1989). As used herein, PCR is considered to be one, but not the only, example of a nucleic acid polymerase reaction method for amplifying a nucleic acid test sample comprising the use of a known nucleic acid as a primer and a nucleic acid polymerase to amplify or generate a specific piece of nucleic acid.

The term “primer” refers to a nucleic acid capable of acting as a point of initiation of synthesis along a complementary strand when conditions are suitable for synthesis of a primer extension product. The synthesizing conditions include the presence of four different bases and at least one polymerization-inducing agent such as reverse transcriptase or DNA polymerase. These are present in a suitable buffer, which may include constituents which are co-factors or which affect conditions such as pH and the like at various suitable temperatures. A primer is preferably a single strand sequence, such that amplification efficiency is optimized, but double stranded sequences can be utilized.

The term “probe” refers to a nucleic acid that hybridizes to a target sequence. In some embodiments, a probe includes about eight nucleotides, about 10 nucleotides, about 15 nucleotides, about 20 nucleotides, about 25 nucleotides, about 30 nucleotides, about 40 nucleotides, about 50 nucleotides, about 60 nucleotides, about 70 nucleotides, about 75 nucleotides, about 80 nucleotides, about 90 nucleotides, about 100 nucleotides, about 110 nucleotides, about 115 nucleotides, about 120 nucleotides, about 130 nucleotides, about 140 nucleotides, about 150 nucleotides, about 175 nucleotides, about 187 nucleotides, about 200 nucleotides, about 225 nucleotides, and about 250 nucleotides. A probe can further include a detectable label. Detectable labels include, but are not limited to, a fluorophore (e.g., Texas-Red®, Fluorescein isothiocyanate, etc.,) and a hapten, (e.g., biotin). A detectable label can be covalently attached directly to a probe oligonucleotide, e.g., located at the probe's 5′ end or at the probe's 3′ end. A probe including a fluorophore may also further include a quencher, e.g., Black Hole Quencher™, Iowa Black™, etc.

The terms “nucleic acid” and “polynucleotide” are used interchangeably herein to describe a polymer of any length, e.g., greater than about 10 bases, greater than about 100 bases, greater than about 500 bases, greater than 1000 bases, usually up to about 10,000 or more bases composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Nucleic acids can include genomic sequence, cDNA, mRNA, introns, exons, leader sequences, and regulatory sequences.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “melting temperature” or “Tₘ” refers to the temperature where the DNA duplex will dissociate and become single stranded. Thus, Tm is an indication of duplex stability.

The terms “hybridize” or “hybridization,” as is known to those of ordinary skill in the art, refer to the binding or duplexing of a nucleic acid molecule to a particular nucleotide sequence under suitable conditions, e.g., under stringent conditions. The term “stringent conditions” (or “stringent hybridization conditions”) as used herein refers to conditions that are compatible to produce binding pairs of nucleic acids, e.g., surface bound and solution phase nucleic acids, of sufficient complementarity to provide for a desired level of specificity in an assay while being less compatible to the formation of binding pairs between binding members of insufficient complementarity to provide for the desired specificity. Stringent conditions are the summation or combination (totality) of both hybridization and wash conditions.

The term “stringent assay conditions” as used herein refers to conditions that are compatible to produce binding pairs of nucleic acids, e.g., probes and targets, of sufficient complementarity to provide for the desired level of specificity in the assay while being incompatible to the formation of binding pairs between binding members of insufficient complementarity to provide for the desired specificity. The term stringent assay conditions refers to the combination of hybridization and wash conditions.

A “stringent hybridization” and “stringent hybridization wash conditions” in the context of nucleic acid hybridization (e.g., as in array, Southern or Northern hybridizations) are sequence dependent, and are different under different environmental parameters. Stringent hybridization conditions that can be used to identify nucleic acids as described herein can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions determine whether a nucleic acid is specifically hybridized to a probe. Wash conditions used to identify nucleic acids may include, e.g.: a salt concentration of about 0.02 M at pH 7 and a temperature of about 20° C. to about 40° C.; or, a salt concentration of about 0.15 M NaCl at 72° C. for about 15 minutes; or, a salt concentration of about 0.2×SSC at a temperature of about 30° C. to about 50° C. for about 2 to about 20 minutes; or, the hybridization complex is washed twice with a solution with a salt concentration of about 2×SSC containing 1% SDS at room temperature for 15 minutes and then washed twice by 0.1×SSC containing 0.1% SDS at 37° C. for 15 minutes; or, equivalent conditions. Stringent conditions for washing can also be, e.g., 0.2×SSC/0.1% SDS at 42° C. See Sambrook, Ausubel, or Tijssen (cited below) for detailed descriptions of equivalent hybridization and wash conditions and for reagents and buffers, e.g., SSC buffers and equivalent reagents and conditions.

As used herein, the term “genotype” means a sequence of nucleotide pair(s) found at one or more sites in a locus on a pair of homologous chromosomes in an individual. Genotype may refer to the specific sequence of the gene.

As used herein the term “oligomer inhibitor” means an inhibitor that has the ability to block primer or probe annealing to a nucleic acid sequence. The inhibitor may be a polynucleotide designed to competitively inhibit binding of primer or probe to cDNA that is similar but not identical to the target template sequence. The “oligomer inhibitor” may contain a complementary or about complementary sequence to a non-specific target sequence. A polynucleotide oligomer inhibitor may vary in size from about 3 to about 100 nucleotides, about 5 to about 50 nucleotides, about 7 to about 20 nucleotides, about 8 to about 14 nucleotides.

As used herein, the term “about” modifying the quantity of an ingredient, parameter, calculation, or measurement in the compositions described herein or employed in the methods as described herein refers to variation in the numerical quantity that can occur, for example, through typical measuring and liquid handling procedures used for making DNA, probes, primers, or solutions in the real world; through inadvertent error in these procedures; through differences in the manufacture, source, or purity of the ingredients employed to make the compositions or carry out the methods; and the like without having a substantial effect on the chemical or physical attributes of the compositions or methods as described herein. The term about also encompasses amounts that differ due to different equilibrium conditions for a composition resulting from a particular initial mixture. Whether or not modified by the term “about” the claims include equivalents to the quantities.

### DETAILED DESCRIPTION OF THE DISCLOSURE

Eleven families with apparent inherited predisposition to PPB as evidenced by two or more relatives with PPB, lung cysts and/or cystic nephroma were analyzed for genetic alterations. DNA marker linkage studies on four families mapped a PPB susceptibility locus to a 7 Mb region of distal chromosome 14q. A total of 49 individuals were included in DNA marker linkage studies. Sequence analysis identified heterozygous DICER1 mutations in peripheral blood leukocytes from these four families and seven additional families.

DICER1 polypeptide, a ribonuclease III enzyme, has the critical role of cleaving precursor microRNAs (miRNA) and small interfering RNAs (siRNA) into their mature (active) forms. miRNAs are the functional elements of a relatively newly discovered, yet highly conserved cellular apparatus for regulating protein expression. DICER1-processed mature miRNAs can bind specific mRNA sequences and target them for destruction or inhibiting translation. miRNA regulatory processes are very important in organ development, including lung branching morphogenesis, cell cycle control and oncogenesis. It has been postulated that a subgroup of miRNAs act as tumor suppressors. The presence of germline DICER1 mutations in patients with PPB suggests that aberrant miRNA processing can both adversely impact developmentally-timed programs in the lung and confer risk for malignant evolution.

Nucleic acids, Primers, and Probes

This disclosure provides an isolated nucleic acid that comprises a nucleic acid that encodes a portion of a DICER1 polypeptide or that comprises a portion of the DICER1 gene, wherein the nucleic acid comprises a nucleotide position that can be mutated as compared to a reference sequence, wherein when the nucleotide position is mutated a structure or function of DICER1 polypeptide is altered. In some embodiments the isolated nucleic acid excludes the naturally occurring full length genomic sequence such as provided in Tables 3 and 4 and/or from subjects with no history of PPB or other cancers, one or more full length naturally occurring exon sequences such as provided in Tables 3 and 4 and/or from subjects with no history of PPB or other cancers, or a full length naturally occurring mRNA sequence such as provided in Tables 3 and 4 and/or from subjects with no history of PPB or other cancers.

In some embodiments, an isolated nucleic acid that specifically hybridizes to the isolated nucleic acid, wherein the nucleic acid preferentially hybridizes to the sequence comprising the mutation at the nucleotide position as compared to a corresponding sequence that does not have the mutation at that nucleotide is provided. In other embodiments, an isolated nucleic acid that specifically hybridizes to the isolated nucleic acid sequence, wherein the nucleic acid preferentially hybridizes to the sequence without the mutation at the nucleotide position as compared to a corresponding sequence that does have a mutation at the nucleotide position is provided. In some embodiments the reference sequence is all or a portion of the nucleic acid sequence of SEQ ID NO:2.

The gene for DICER1 includes 27 exons, introns and regulatory regions. Mutations can occur within exons, introns, regulatory regions, and at the junction between introns and exons. Mutations can include missense, nonsense, frameshift, deletions, insertions, and stop codons. In some embodiments, the insertions can include from 1 to 21 nucleotides, 1 to 12 nucleotides, 1 to 6 nucleotides or 1 to 3 nucleotides. In some embodiments deletions can be of one or more exonic or intronic regions, or about 1 to 21 nucleotides, 1 to 12 nucleotides, 1 to 6 nucleotides or 1 to 3 nucleotides. In some embodiments the mutations are found at the intron exon splice sites, within introns, or within exons. In some embodiments, the nucleotide position or positions that are mutated are located in an exon selected from the group consisting of exon 9, exon 10, exon 12, exon 14, exon 15, exon 18, exon 21, exon 23 and combinations thereof.

In some embodiments, the mutation results in a loss of function of the DICER1 polypeptide. Loss of function of the DICER1 polypeptide can be determined by assaying for ribonuclease activity or by binding to an antibody that binds to a ribonuclease domain of DICER1. In some embodiments, the mutations are located upstream from the genomic sequences surrounding or encoding one or more ribonuclease domains. In other embodiments, the mutation results in an alteration of the structure of DICER 1 polypeptide, including one or more domains such as the RNase domains.

In another aspect the disclosure provides primers and/or probes useful in the detection of one or more mutations in a nucleic acid sequence comprising a nucleic acid that that encodes a portion of a DICER1 polypeptide or that comprises a portion of the DICER1 gene. Primers or probes can be designed to hybridize to a specific exon and/or intron such as provided in Table 2A. Primers and/or probes can be designed to detect and/or amplify the nucleic acid region surrounding the mutation. In some embodiments, the primers are designed to amplify the mutation as well as 20 to 1000 nucleotides, 20 to 900 nucleotides, 20 to 800 nucleotides, 20 to 700 nucleotides, 20 to 600 nucleotides, 20 to 500 nucleotides, 20 to 400 nucleotides, 20 to 300 nucleotides, 20 to 200 nucleotides, 20 to 100 nucleotides, and 20 to 50 nucleotides surrounding the site of the mutation. In specific embodiments, locations for targeting the probes and/or primers are those shown in Table 1.

Primers or probes can be designed to provide for amplification and/or detection of a number of introns and exons including one or more exons selected from exon 9, exon 10, exon 12, exon 14, exon 15, exon, 18, exon 21, exon 23 and combinations thereof. Primers or probes can be designed to provide for amplification and/or detection of more than one exon including, but not limited to, from about exon 9 to about exon 23, from about exon 9 to exon 21, from about exon 9 to about exon 18, from about exon 9 to about exon 15, from about exon 9 to about exon 14, from about exon 9 to about exon 12, from about exon 9 to about exon 10, and combinations thereof.

In specific embodiments, one or more primers and/or probes have a sequence selected from the group consisting of SEQ ID NO:6 to SEQ ID NO:80 including the sequences in tables 2A, 2B, 2C, and Table 8.

In some embodiments, the isolated nucleic acid sequence has about 80 to 100% sequence identity to a reference sequence including every percentage in between 80 and 100%. Reference sequences can include a full length mRNA or genomic sequence as provided in SEQ ID NO:2 or can be a full length intron or exon sequence. Naturally occurring allelic variants of the DICER1 gene can exist without affecting the function of the DICER1 polypeptide. Primers and probes can be designed to account for variants in the DICER1 genomic sequence.

Antibodies or functional assays can also be used to detect the presence or absence of a functioning DICER1 polypeptide in a cell sample. Ribonuclease assays on tissue samples can be conducted using standard methods. Immunochemical staining or lack thereof can be conducted using an antibody, such as antibody that binds to a ribonuclease domain of DICER1, can also be used to determine the presence or absence of a functional DICER1 polypeptide in a cell. Antibodies can be prepared directed to one or more of the polypeptides that are produced as a result of the mutations of the Dicer gene as described herein using standard methods.

The isolated nucleic acids, primers, probes, and antibodies can be detectably labeled. In some embodiments, the label is selected from the group consisting of Texas-Red®, fluorescein isothiocyanate, FAM, TAMRA, Alexa flour, a cyanine dye, a quencher, and biotin.

Methods and Kits

This disclosure provides reagents, methods, and kits for determining the presence and/or amount of: a) at least one mutation in a DICER 1 gene; b) mutant mRNA encoding DICER1 polypeptide; and/or c) mutant DICER1 polypeptide in a biological sample.

Methods include a method of detecting the presence of a mutation in a DICER1 nucleic acid sequence, comprising: isolating a nucleic acid that comprises a nucleic acid that encodes a portion of a DICER1 polypeptide or that comprises a portion of the DICER1 gene, wherein the nucleic acid comprises a nucleotide position that can be mutated as compared to a reference sequence, wherein when the nucleotide position is mutated a function of DICER1 polypeptide is decreased and/or the one or more RNAse domains are altered and sequencing the isolated nucleic acid to determine whether the nucleotide in the nucleotide position is mutated as compared to the reference sequence. Another method provides a method of detecting the presence of a mutation in a DICER1 nucleic acid sequence, comprising: contacting the nucleic acid that comprises a nucleic acid that encodes a portion of a DICER1 polypeptide or that comprises a portion of the DICER1 gene with a primer or probe under conditions suitable for hybridization and/or amplification, wherein the nucleic acid comprises a nucleotide position that can be mutated as compared to a reference sequence, wherein when the nucleotide position is mutated a function of DICER1 polypeptide is decreased and/or the one or more RNAse domains are altered, and determining whether the nucleic acids hybridize to one another and/or determining the size and/or sequence of the amplified region.

In other embodiments, a method comprises determining whether the nucleic acids hybridize to one another comprises determining whether a mismatch is present by contacting the hybridized sample with an agent that cleaves at the site of a mismatch, and identifying the size of any of the products of the cleavage reaction, wherein if a mismatch is present a cleavage product is detected.

In some embodiments, the method involves detecting a germline mutation using an array or probe designed to distinguish mutations in a DICER1 gene. Mutations include insertions, deletions, and substitutions. In some embodiments, substitutions result in the formation of stop codons. In other embodiments, insertions or deletions result in frameshift or missense mutations. Probes or cDNA oligonucleotides that detect mutations in a nucleic acid sequence can be designed using methods known to those of skill in the art and as described above.

In some embodiments, mutations are identified as those that lead to a decrease in expression of DICER1. In some embodiments, the DICER1 mutation is proximal to DICER1's two carboxy-terminal RNase III functional domains. In some embodiments, the mutation is located in the helicase domain, dsRNA binding fold, the Pax domain and/or in one or more introns before one of the RNAse domains. In some embodiments, the mutation is a missense, frameshift, or stop codon mutation. In an embodiment, the mutation results in a truncation of the DICER1 polypeptide. In some embodiments, the mutations are one or more or all the mutations shown in Table 1.

In embodiments, the methods and kits may provide restriction enzymes and/or probes that can detect changes to the restriction fragments as a result of the presence of at least one mutation in the gene sequence encoding DICER1. The publically available human genome sequence can be used to generate a RFLP map.

In other embodiments, the method excludes detection of at least one mutation in DICER1 that does not result in a change to the DICER1 polypeptide or mRNA such as the change at position 5558 from T to C or position 4154 from G to A. In some embodiments, mutations that do not result in a loss of function of the DICER1 polypeptide or mRNA are excluded.

In another aspect, a highly sensitive and specific quantitative PCR assay to detect one or more mutant mRNAs of the DICER1 gene is provided. In embodiments, the methods and kits provide for primers and probes that can detect the presence of at least one mutation in the mRNA and/or detect an alteration in size or sequence of mRNA (such as in the case of truncation). In embodiments, the primers are those shown in Table 2A, 2B, 2C, and Table 8. In some embodiments, primers are designed to hybridize within a certain temperature range and may also include other sequences such as universal sequencing sequences.

In some embodiments, the target sequence of the primer/probe sets include those that are complementary to mature coding sequence including exons at the 3′ end encoding the ribonuclease domains. Those primer/probes can act as a positive control to detect full length transcripts that encode active DICER polypeptide. In some embodiments, the primers and probes complementary to the 3′ untranslated region are excluded as positive controls in order to avoid spurious detection of degraded mRNA and to enhance the correlation between the mRNA that is measured by this assay and the protein that is actually expressed.

In some embodiments, the assay can exploit two modifications of probe-based RT-PCR: molecular beacons (MB) and locked nucleic acids (LNA). In specific embodiments, one or more primers and/or probes have a sequence selected from the group consisting of SEQ ID NO:6 to SEQ ID NO:80 including the sequences in tables 2A, 2B, 2C, and Table 8.

In some embodiments, the kit can include one or more probes and/or primer attached to a solid substrate. In some embodiments, an array can comprise one more of the sequences found in Tables 2A, B, and C. In some embodiments, the array or kit includes detection of expression of the growth factor genes. In some embodiments, the array or kit excludes detection of a gene selected from the group consisting of actin, gapdh, aldolase, hexokinase, cyclophilin and combinations thereof. In some embodiments, the array or kit detects less than 2000 genes, less than 1000 genes, less than 500 genes, less than 200 genes, less than 100 genes, less than 50 genes, and less than 10 genes.

In some embodiments, the methods and kits provide reagents for detection of the presence or absence of the DICER polypeptide. In some embodiments, the reagents include an antibody that can detect full length DICER polypeptide in cells. In other embodiments, an antibody can detect polypeptides that have an alteration in one or more domains of the DICER polypeptide including the RNase domains. The antibodies can be detectably labeled. Detectable labels include fluorescent labels, radioactive isotope labels, and polypeptide labels including enzymes or molecules like biotin. The methods of detection involve immunohistochemical or radiological detection of DICER1 polypeptide or altered DICER polypeptide in tumor tissue.

The kit can establish patterns of DICER1 expression that may be associated with protection from, or pathogenesis of many diseases, including PBB and associated PBB diseases such as cystic nephroma, renal cysts, thyroid carcinoma, intestinal polyps, leukemia, ovarian germ cell tumors, testicular germ cell tumors, ovarian dysgerminoma, testicular seminoma, hepatic hamartomas, nasal chondromesenchymal hamartoma, Wilms tumor, rhabdomyosarcoma, synovial sarcoma, Sertoli-Leydig tumors, medulloblastoma, glioblastoma multiforme, primary brain sarcoma, ependymoma, neuroblastoma, and neurofibromatosis Type I. The presence of a DICER1 mutation can be used to prognosticate risk of malignancy, identify appropriate treatment based on the risk of malignancy, and to diagnose one or more of the above tumors.

The disclosure provides a method of determining the diagnosis or prognosis of a cancer comprising: determining whether the nucleic that comprises a nucleic acid that encodes a portion of a DICER1 polypeptide or that comprises a portion of the DICER1 gene has the reference sequence or the mutated sequence. In embodiments, the expression or decrease in expression in a cell sample or cell type can be determined by PCR analysis, hybridization analysis, in situ analysis using hybridization or antibody detection methods.

In some embodiments, the cancer is selected from the group consisting of PBB, cystic nephroma, renal cysts, thyroid carcinoma, intestinal polyps, leukemia, ovarian germ cell tumors, testicular germ cell tumors, ovarian dysgerminoma, testicular seminoma, hepatic hamartomas, nasal chondromesenchymal hamartoma, Wilms tumor, rhabdomyosarcoma, synovial sarcoma, Sertoli-Leydig tumors, medulloblastoma, glioblastoma multiforme, primary brain sarcoma, ependymoma, neuroblastoma, and neurofibromatosis Type I.

In other embodiments, the cancer has a mesenchymal and epithelial component, and a cell sample may include one or both cell types. Other cancers that have an epithelial and mesenchymal component include carcinosarcoma and/or sarcomatoid cancers of the breast, uterus, lung, and gastrointestinal tract, malignant mesothelioma, sex chord stromal tumors, and ameloblastoma. In some embodiments, the cancer can also be characterized by having an epithelial to mesenchymal transition by identifying a change in other markers such as e-cadherins or based on histopathology of a tumor sample. Such transitions are also associated with an increased risk of metastasis.

In some embodiments, once a cancer is diagnosed or a cyst is identified in a patient other family members may also be examined for the presence or absence of mutation in DICER1.

In some embodiments, after detection of one or mutations in DICER1 is detected, a treatment is selected and administered to the patient. A method of treating a cancer, comprising administering to a tumor cell a nucleic acid that has at least 80% sequence identity to the nucleic acid sequence that encodes a DICER1 polypeptide having the sequence of SEQ ID NO:1, wherein the polypeptide has DICER1 activity. In some embodiments, the cancer is selected from the group consisting of PBB, cystic nephroma, renal cysts, thyroid carcinoma, intestinal polyps, leukemia, ovarian germ cell tumors, testicular germ cell tumors, ovarian dysgerminoma, testicular seminoma, hepatic hamartomas, nasal chondromesenchymal hamartoma, Wilms tumor, rhabdomyosarcoma, synovial sarcoma, Sertoli-Leydig tumors, medulloblastoma, glioblastoma multiforme, primary brain sarcoma, ependymoma, neuroblastoma, and neurofibromatosis Type I. In some embodiments, the nucleic acid is present in an expression vector.

### Example 1

Methods and Study Subjects

Families were ascertained through the International PPB Registry (www.ppbregistry.org). All research subjects provided written consent for molecular and family history studies as approved by the Human Research Protection Office at Washington University. St. Louis, Mo. Blood and saliva specimens were collected as a source of genomic DNA. Detailed family histories were obtained by an experienced genetic counselor. All PPB cases were centrally reviewed and whenever possible, medical records and pathology materials were obtained to confirm other reported tumors. Eleven multiplex families (those with more than one “affected” member) were investigated. Individuals were classified as “affected” if they had either PPB, lung cysts, cystic nephroma or embryonal rhabdomyosarcoma. (Priest et al.)

DNA Marker Linkage Analysis and Mapping

Four families were selected for linkage studies based on the availability of DNA specimens from affected members of the kindreds and family structure. Genotyping was performed on 49 individuals with Affymetrix Genome-wide Human SNP Arrays v6.0 (Affymetrix, Santa Clara, Calif.). (Hill). Genomic DNA samples from each of the 49 individuals was fragmented, amplified and labeled for hybridization. Data files containing genotype calls for each sample were exported using the Affymetrix GeneChip Genotyping Console Software. Genotypes were generated with the Birdseed algorithm using default settings.

A subset of the over 900,000 polymorphic markers represented on the SNP array was selected for linkage analysis based on pairwise measurements of linkage disequilibrium (LD) and estimates of heterozygosity. We used Affymetrix 6.0 data from 30 CEPH (Caucasian) families as a reference data set (available at the Affymetrix website). In short, r² was calculated for each pair of adjacent markers. Because marker selection was intended to minimize the use of markers in high LD which may contribute to Type I error, we were conservative with our approach. For marker pairs showing an r²>0.1, the marker with the least heterozygosity was discarded. The method was reiterated sequentially for all markers on each chromosome using a one Mb sliding window. 4117 SNPs were ultimately selected for linkage analysis.

Linkage files and genotypes from four families were then imported into the easyLinkage Plus program (v5.08). Markers with call rates <95% (n=281) were removed. Mendelian error-checking was performed using the Pedcheck program and markers creating Mendelian errors (n=110) were removed from the data set. Multipoint non-parametric and parametric linkage analyses were then performed using the Genehunter v.2.1r5 algorithm combining the data from the four families. The parametric analysis assumed autosomal dominant inheritance and obligate heterozygotes were modeled as unaffected, unknown, and affected. All three of these parametric models yielded similar results; LOD scores did not vary by more than 0.3. Penetrance was assumed at 0, 0.25 and 0.25 for wild type/wild type, wild type/mutant, and mutant/mutant genotypes respectively. The disease allele frequency was set at 0.001.

The candidate region suggestive of linkage on distal 14q was further evaluated by creating haplotypes using an expanded set of ˜7000 Affy 6.0 markers from region surrounding the linkage peak. Haplotypes generated from this analysis were imported into Haplopainter for easy visualization. The minimum overlap for the PPB susceptibility locus was inferred based on recombination events visualized in affected individuals from each of the four families.

Sequence Analysis of DICER1, a PPB Candidate Gene

DICER1 sequences were extracted from the public draft human genome database (ref sequence NM_{—}177438; build 36.1; Table 4, SEQ ID NO:2) and used as a reference sequence for assembly and primer construction. The genomic sequence was obtained from position hg18_chr14:94621318-94694512_rev. Primers to amplify all of the coding exons including intron-exon boundaries were designed either using the Primer 3 or the UCSC exon primer program and are shown in Table 2A. (Kent, W. J. “BLAT—the BLAST-like alignment tool.” Genome Res. 12 (2002): 656-64; Kent, W. J. Genome Res. 12 (2002): 996; Kuhn, R. M., et al. “The UCSC Genome Browser Database: update 2009.” Nucleic Acids Res. (2008).). Universal M13 tails were added to the 5′ ends of the PCR primers to facilitate sequence analysis. All primers are listed 5′ to 3′. Table 2A shown below.

| NAME | LEFT_PRIMER | RIGHT_PRIMER | SIZE |
|---|---|---|---|
| Exon2 | TCAAATCCAATTACCCAGCAG | GCAATGAAAGAAACACTGGATG | 358 |
| | (SEQ ID NO: 16) | (SEQ ID NO: 42) | |
| Exon3 | TCTGCCAGAAGAGATTAAATGAG | TTTTGTAAATTTATTGGAGGACG | 429 |
| | (SEQ ID NO: 17) | (SEQ ID NO: 43) | |
| Exon4 | AAATCAGACAACCAAGGCTACAG | TTTTGGAGGATAACCTTGGAAC | 390 |
| | (SEQ ID NO: 18) | (SEQ ID NO: 44) | |
| Exon5 | TTTAATATTCATTCATTCATACACTGC | TTGTCGTCAAGACATGCTTTC | 518 |
| | (SEQ ID NO: 19) | (SEQ ID NO: 45) | |
| Exon6 | GAATTCTTACTCTTGCCCATTCC | TAGTGGCATTTCCACCAAAC | 437 |
| | (SEQ ID NO: 20) | (SEQ ID NO: 46) | |
| Exon7 | GAGCCGCATTAAGCATATTTTC | CCCACTGCTAACATTCTGGC | 395 |
| | (SEQ ID NO: 21) | (SEQ ID NO: 47) | |
| Exon8 | TCACATCACAACACAGGACG | AAATCCCAGTTAAACCCCAC | 614 |
| | (SEQ ID NO: 22) | (SEQ ID NO: 48) | |
| Exon9 | AAATCACTCTACAGCTACCTCATGG | TAAATCACCGTCGCCAAATC | 820 |
| | (SEQ ID NO: 23) | (SEQ ID NO: 49) | |
| Exon10 | TTCCTATGGATACAAAGAATAACAAAG | CATGTGTGTCAGAAATGACAGTTG | 431 |
| | (SEQ ID NO: 24) | (SEQ ID NO: 50) | |
| Exon11 | AACTTTTATTGCTGCACGATACTG | AGCAGGTTACTTTGGAGTACTGAAG | 760 |
| | (SEQ ID NO: 25) | (SEQ ID NO: 51) | |
| Exon12 | TGAACATGTAGATGACTACAAAAGC | TCACATTTCAAGTGCTCACC | 777 |
| | (SEQ ID NO: 26) | (SEQ ID NO: 52) | |
| Exon13 | AAGTGTTCATGGTGCATGATTC | TTTTACTAGGCAGGACTTTTAAAGATG | 585 |
| | (SEQ ID NO: 27) | (SEQ ID NO: 53) | |
| Exon14 | AAGCTGTGAATCGGAGAAAG | TTTGCAGTCCAGCTCATATTG | 760 |
| | (SEQ ID NO: 28) | (SEQ ID NO: 54) | |
| Exon15 | TCTAGTGGAGAAATAGAAGAGGCAC | TAAGAAGTGTCATGCCTCGG | 468 |
| | (SEQ ID NO: 29) | (SEQ ID NO: 55) | |
| Exon16-17 | TTTTAGTAGAGACGAGGTTTCACC | GAAAGCATCATTTCTGTTCTGAAG | 754 |
| | (SEQ ID NO: 30) | (SEQ ID NO: 56) | |
| Exon18 | TTTGTGTGCAAAGCATCTCC | TGTAAAGGTGCCATTTAGCTTC | 589 |
| | (SEQ ID NO: 31) | (SEQ ID NO: 57) | |
| Exon19 | TTTGTGATATATTAATGGGCCAAG | ATTGCACTTGAGGGATTCTTACC | 582 |
| | (SEQ ID NO: 32) | (SEQ ID NO: 58) | |
| Exon20 | TCTCACTCCAACTGTTATGGCTTA | TTGGCCCATTAATATATCACA | 776 |
| | (SEQ ID NO: 33) | (SEQ ID NO: 59) | |
| Exon21_1 | GAGTACATTCATCGCTGGGC | AATTGCTGTTGCTCTCAGCC | 508 |
| | (SEQ ID NO: 34) | (SEQ ID NO: 60) | |
| Exon21_2 | ACTGCAAACCACTTTCAGGC | ACAAGCAGGAAATACCCGTG | 501 |
| | (SEQ ID NO: 35) | (SEQ ID NO: 61) | |
| Exon22 | AGAAATTTGCCTCCATCAAA | AAAGCATAGAATATGTGGGAATT | 725 |
| | (SEQ ID NO: 36) | (SEQ ID NO: 62) | |
| Exon23_1 | CAGGGCTTCCACACAGTCC | AACCCTTGCTTTTATTGAGTTTC | 574 |
| | (SEQ ID NO: 37) | (SEQ ID NO: 63) | |
| Exon23_2 | TACAAGGCCAACACGATGAG | AAACTGTGGTGTTGACACGG | 571 |
| | (SEQ ID NO: 38) | (SEQ ID NO: 64) | |
| Exon24 | TGCCGTCAGAACTCTGAAAC | TGTGGGGATAGTGTAAATGCTTC | 403 |
| | (SEQ ID NO: 39) | (SEQ ID NO: 65) | |
| Exon25-26 | TGAACTTTTCCCCTTTGATG | TGGACTGCCTGTAAAAGTGG | 450 |
| | (SEQ ID NO: 40) | (SEQ ID NO: 66) | |
| Exon27 | TCTGCCTTCAATTCATTCCA | CCTGTCTGTCGGGGGTATG | 448 |
| | (SEQ ID NO: 41) | (SEQ ID NO: 67) | |

PCR reactions were performed using genomic DNA from the probands for each of the 11 multiplex families. Taq polymerase was used with 1.5 microliter of primer (10 nmol dilution) in total reaction volume of 50 microliter. The following cycling conditions were used: 95° 5 min. then 14 cycles at with 30 sec at 95°; 45 sec at 63°; 45 sec at 70°, then 20 cycles at 30 sec at 94°; 45 sec at 56°; and 45 sec at 70°, and then hold at 70° for 10 minutes, followed by holding at 4°.

The resultant products were purified by PEG/5 M NaCl/Tris precipitation and directly sequenced using BigDye Terminator chemistry (v3.1 Applied Biosytems, Valencia Calif.) and the ABI3730 sequencer (Applied Biosystems). Exon 1 (noncoding) was analyzed in one family using primers shown in Table 2B. The SIFT algorithm was used to assess significance of the missense change identified in one family. The sequence traces were assembled and scanned for variations using Sequencer version 4.8 (Gene Codes, Ann Arbor, Mich.). All variants were confirmed by bi-directional sequencing and queried against the NCBI dbSNP Build 128 database. Pyrosequencing™ was performed to assess the frequency of one missense DICER1 sequence alteration in 360 cancer-free controls (siteman/wustl.edu/internal.aspx) (Table 2B).

TABLE 2B
Table 2B: Primers and conditions use for amplification of DICER1
sequences and Primers for Pyrosequencing

| | | | | Ampli- | | MgCl₂ |
|---|---|---|---|---|---|---|
| | Forward Primer | Reverse Primer | | con | No. | Concen- |
| Exon | (SEQ ID NO: 68 | (SEQ ID NO: 69 | Annealing Temp | Size | Cycles | tration |
| 1 | 5′ aatcacaggctcgctctcat 3′ | 5′ gtctccacctccgctgct 3′ | 63° C. | 762 bp | 30 | 1.5 mM* |
| Sequencing DICER1 4930T → G | | | | | | |
| | Forward Primer** | Reverse Primer | Sequencing primer | | | |
| | (SEQ ID NO: 70) | (SEQ ID NO: 71) | (SEQ ID NO: 72) | | | |
| | 5′gggaaagcagtccatttcttacg3′ | 5′accttcagccccagtgaaca3′ | 5′tcagccccagtgaac3′ | | | |

*plus 1.3 M Betaine
**biotinylated

DICER1 Expression Analysis

RNA was extracted from lymphoblastoid cell lines available from affected members of five families. RNA and protein were extracted from lymphoblasts for RT-PCR and Western blot analysis of DICER1. RT-PCR was performed to assess regions of family-specific mutations and the resultant products were directly sequenced (Table 2C).

TABLE 2C
Primers for RT-PCR analysis of DICER1 mutations

| | | | Annealing | Amplicon | No. |
|---|---|---|---|---|---|
| Assay | Forward Primer | Reverse Primer | Temp | Size | Cycles |
| Family B, exon | CCTGATCAGCCCTGTTACCT | CCTGATCAGCCCTGTTAC | 59° C. | 186 bp | 35 |
| 15 mutation | (SEQ ID NO: 73) | CT | | | |
| | | (SEQ ID NO: 77) | | | |
| Family D, exon | TGTGGAAAGAAGATACACAGCA | TTGGTCTCATGTGCTCGA | 60° C. | 201 bp | 35 |
| 9 mutation | GTTG | AA | | | |
| | (SEQ ID NO: 74) | (SEQ ID NO: 78) | | | |
| Family L, exon | CACCTCTTCGAGCCTCCATTG | GGGCTGATCAGGTCTGGG | 63° C. | 284 bp | 35 |
| 14 mutation | (SEQ ID NO: 75) | ATA | | | |
| | | (SEQ ID NO: 79) | | | |
| Family G, exon | CACCTCTTCGAGCCTCCATTG | GGGCTGATCAGGTCTGGG | 63° C. | | |
| 14 inseretion | (SEQ ID NO: 76) | ATA | | | |
| | | (SEQ ID NO: 80) | | | |

1.5 mM MgCl for all RT-PCR reactions

DICER1 immunohistochemistry was performed on formalin-fixed paraffin embedded (FFPE) samples of PPB tumor tissue from children of 10 of 11 families. Tumor tissues were stained with a commercial rabbit polyclonal antibody raised to a peptide sequence that maps to the PAZ domain of DICER1. (HPA000694, rabbit anti-human, Sigma-Aldrich, St. Louis, Mo.) Bronchial and alveolar epithelium served as positive internal tissue controls. We also stained normal lungs obtained at autopsy (range 12 weeks gestation through adulthood) to better understand normal DICER1 expression during development.

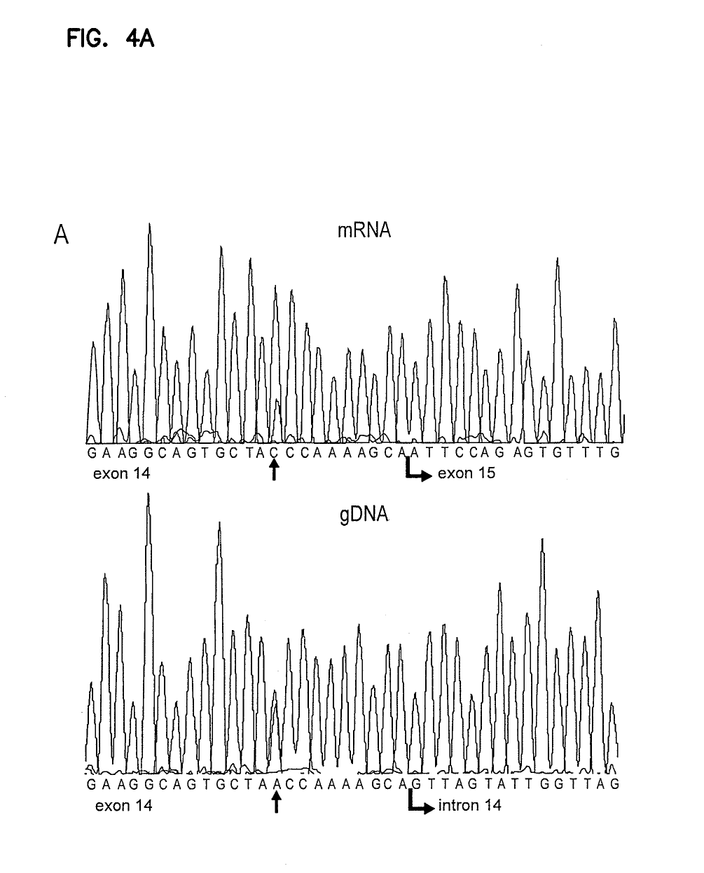

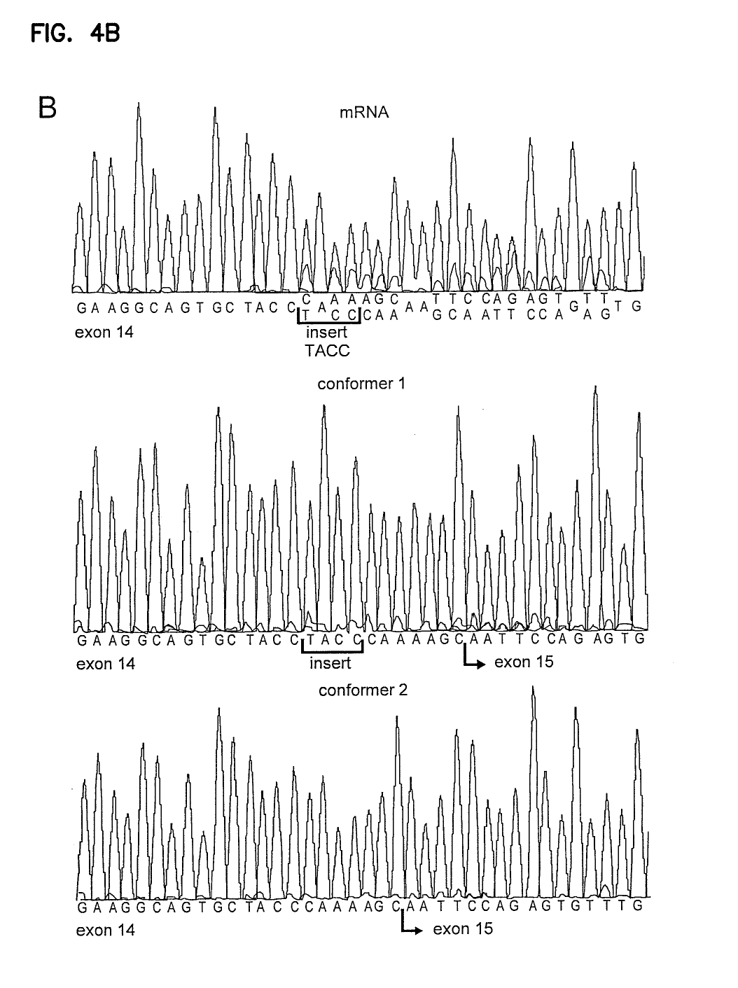

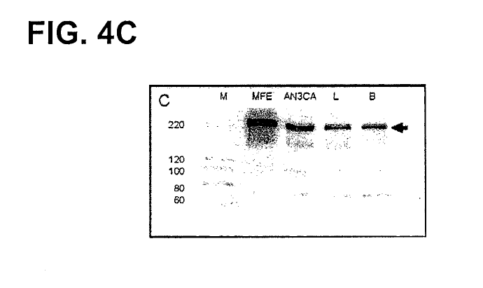

For Western blot analysis, 50 micrograms of cell line lysate run on 4-15% Tris-HCl polyacrylamide gels and transferred to Millipore Immobilon-FL PVDF membrane. DICER1 was detected using an anti-Dicer1 N-terminal antibody raised to a peptide from amino acid 749 to amino acid 798 (13D6, Abeam, Cambrige, MA). Goat anti-mouse IgG-HRP (Santa Cruz Cat# sc-2031) secondary antibody was detected by chemiluminescence (Millipore Immobilon western Chemiluminescent HRP substrate) and BIORAD Chemidoc chemiluminescence. In FIG. 4D, 218 kDa protein (arrow) and the same non-specific bands are seen in lymphoblasts from PPB patients and the MFE and AN3CA control (endometrial cancer) cell lines. Marker (M) sizes in kDa are indicated.

Results

Linkage Analysis Demonstrates a Likely PPB Susceptibility Locus at 14q31-2

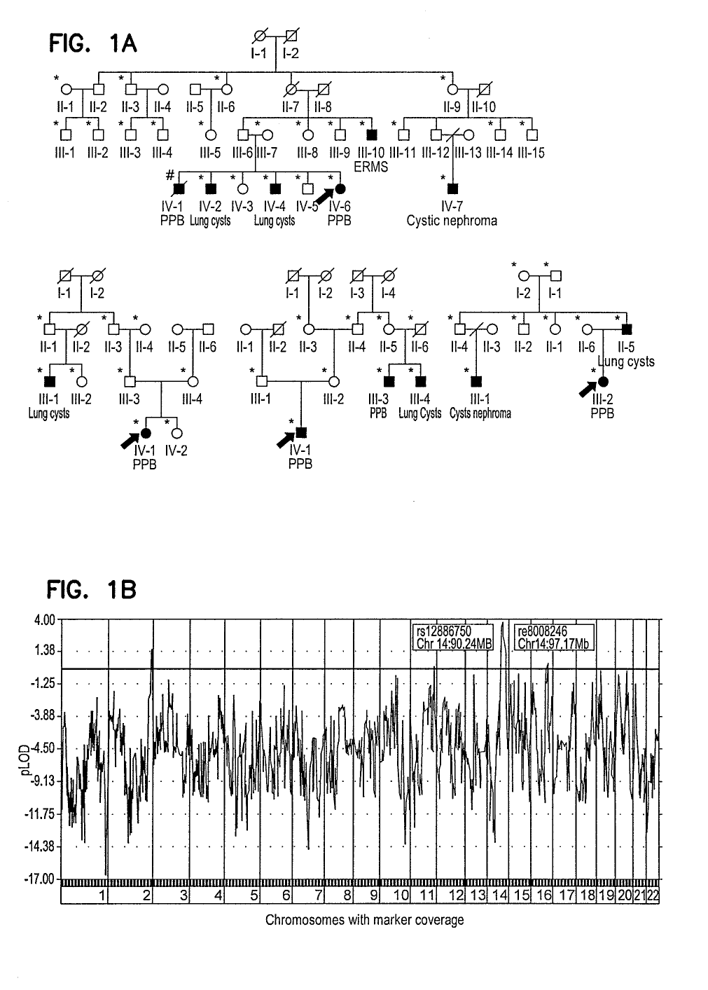

Families included in the DNA marker linkage study are shown in FIG. 1. A total of 68 individuals were genotyped with the Affymetrix 6.0 mapping arrays. Genome-wide non-parametric and parametric multipoint linkage analyses for the four families showed a single peak consistent with linkage on distal chromosome 14 (FIG. 1B). The peak logarithm of odds (LOD) scores from both analyses pointed to a region of linkage on distal 14q. The highest multipoint LOD score for the parametric analysis was 3.71 (FIG. 1B). The peak LOD score was in stark contrast to the rest of the genome for which no interval gave a LOD score greater than 1.40. RFLP analysis of the rs10873449 and rs11160307 markers using FFPE tissue from a deceased affected member of family L (FIG. 1, individual IV-1) revealed transmission of the allele segregating with disease, further supporting linkage to the 14q region.

The candidate region on 14q was further evaluated by creating haplotypes for an expanded set of ˜7000 Affymetrix 6.0 markers spanning the linkage peak (9). The minimum overlap for the PPB susceptibility locus was then inferred based on recombination events visualized in affected individuals from each of the four families (13). The candidate region (flanked by rs12886750 and rs8008246) included 72 annotated genes. (Adie et al.) One gene, DICER1, was a particularly appealing candidate because of its known role in branching morphogenesis of the lung. (Harris et al.) The conditional knock-out of Dicer1 in the mouse lung epithelium results in a cystic lung phenotype that bears striking similarities to type I PPB. (Harris et al.)

Sequence Analysis Identifies Germline Mutations in DICER1 in PPB Families

Sequence analysis of DICER1 in all 11 study families revealed unique germline mutations (FIG. 2A; Table 1). Six families had single base substitutions resulting in stop codons. Three families had insertion or deletion mutations resulting in frameshifts. One family had a single base insertion resulting in a stop codon. For each of these ten families, the predicted mutant protein would be truncated proximal to DICER1's two important carboxy-terminal RNase III functional domains (FIG. 2B). One family (family C) had a single base substitution resulting in a change in from a leucine to an arginine at a position between the two RNase domains.

The probands for families D and L were heterozygous for single base substitutions leading to stop codons (E493X and Y739X, respectively) (FIG. 2B). The DICER1 E493X was present in the germline DNA of the proband's affected father in family D and the Y739X mutation was carried by four other affected individuals in Family L (FIG. 1A). Family B segregated a single base insertion mutation leading to a frameshift (T788Nfs) and family C had a missense mutation resulting in L 1573R (FIG. 2B). The probands from the additional seven multiplex families each carried a truncating mutation (Table 1).

For nine of the PPB families, the observed mutations would result in proteins truncated proximal to DICER1's two carboxy-terminal RNase III functional domains (FIG. 2B). The mutations are therefore almost certainly loss of function defects. The leucine to arginine (L1573R) change in family C is in the region between the two carboxy-terminal RNase III domains (FIG. 2B). The leucine at position 1573 is highly conserved (zebrafish, chicken, rodents and primates). This sequence variant has not been previously reported (NCBI SNP database Build 128) and was not seen in 360 cancer-free controls (16) tested for the 4930T→G substitution by Pyrosequencing™ (Table 2B). The non-polar to charged amino acid change was predicted to not be tolerated based on SIFT analysis (17) and it seems probable that DICER1 function is compromised as a consequence of the amino acid substitution. Taken together, these data provide evidence that DICER1 function is compromised in all families with hereditary PPB.

TABLE 1
Germline DICER1 mutations identified in PPB families.

| Family | | | Predicted amino | Mutant RNA | |
|---|---|---|---|---|---|
| ID | Mutation | Exon | acid change | detection | DICER1 IHC |
| A | 3012C→T | 18 | R934X | Not done | Loss of DICER1 |
| | | | | | staining in tumor |
| | | | | | associated |
| | | | | | epithelium |
| B | 2574insA | 15 | T788Nfs | Reduced | Slides not |
| | | | | | available |
| C | 4930T→G | 23 | L1573R | Not done | Loss of DICER1 |
| | | | | | staining in tumor |
| | | | | | associated |
| | | | | | epithelium |
| D | 1689G→T | 9 | E493X | Reduced | Loss of DICER 1 |
| | | | | | staining in tumor |
| | | | | | associated |
| | | | | | epithelium |
| E | 2092insA | 12 | Y627X | Not done | Loss of DICER1 |
| | | | | | staining in tumor |
| | | | | | associated |
| | | | | | epithelium |
| F | 1866- | 10 | M552Vfs | Not done | NA, Type III PPB |
| | 1867delAT | | | | |
| G | 2430insTACC | 14 | P740Lfs | Reduced | Retained DICER1 |
| | | | | | staining in tumor |
| | | | | | associated |
| | | | | | epithelium; |
| | | | | | no cambium layer |
| | | | | | seen |
| H | 3722C→A | 21 | Y1170X | Not done | NA, Type III PPB |
| I | 1812C→T | 10 | R534X | Not done | Loss of DICER1 |
| | | | | | staining in tumor |
| | | | | | associated |
| | | | | | epithelium |
| L | 2429C→A | 14 | Y739X | Reduced | NA, Type III PPB |
| X | 2204C→T 12 | 12 | R656X | Not done | Loss of DICER1 |
| | | | | | staining in tumor |
| | | | | | associated |
| | | | | | epithelium |

NA, not analyzed (if no cell line was available).
No data because the 13D6 antibody was generated with a peptide antigen C-terminal to the mutation in these families and thus does not provide for detection of the predicted truncations NM177438 was used as the reference sequence for the bases. The amino acid numbering begins with the Kozak sequence.

Marked Reduction in DICER1 Mutant mRNA in Lymphoblastoid Cell Lines from Probands

Lymphoblastoid cell lines were available from affected members from four families (B, D, G and L) carrying mutations that would result in premature stop codons and truncated proteins (Table 1). RNA and protein from lymphoblasts were assessed using RT-PCR and Western blot analysis (8). Direct sequencing of the regions of the DICER1 transcript harboring the family-specific mutations (Table 2C) revealed marked reductions in the levels of mutant mRNA, suggestive of nonsense-mediated decay (26, 27). Reproducible differences in the relative peaks heights corresponding to mutant and wild-type mRNAs were seen for all four mutations.

The single base substitution (2429C→A) in exon 14 in family L was detectable, but at a low level (FIG. 4A). The four base insertion (2430insTACC) mutation seen in exon 14 in family G, represented approximately one-quarter of the DICER1 transcripts based on relative peak heights. (FIG. 4B). The significant reduction in mutant mRNA in lymphoblastoid lines from the four mutation carriers investigated suggests the mutation carriers may have reduced transcripts in a range of somatic tissues and potentially reduced DICER1 protein levels.

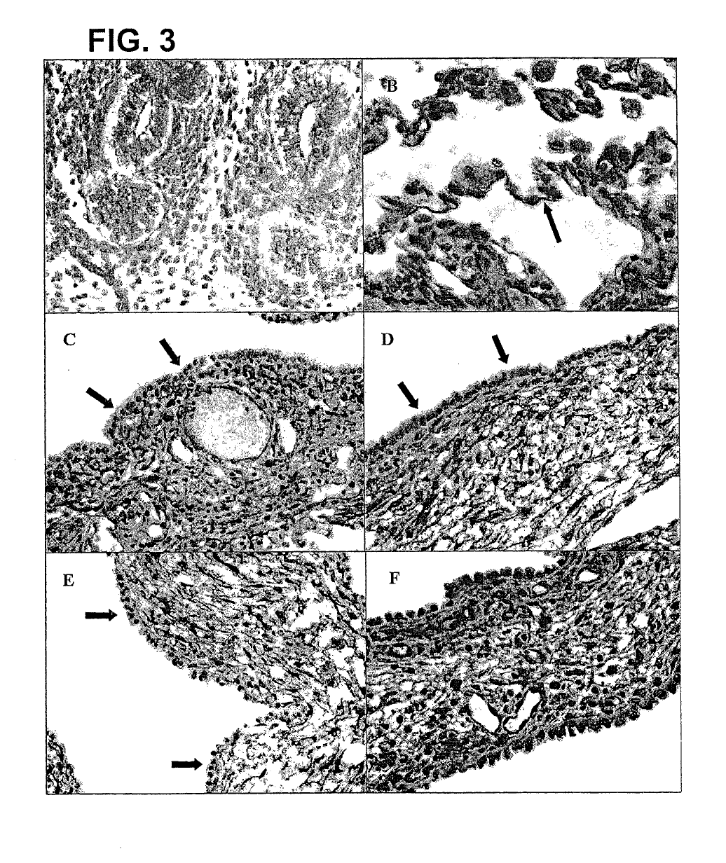

To determine whether development of PPB was associated with loss of DICER 1, human tumors were assessed for DICER1 protein by immunohistochemistry on formalin-fixed sections of PPB tumor tissue (HPA000694, rabbit anti-human, Sigma-Aldrich, St. Louis, Mo.). Tumor slides were available from children with PPB in 10 of 11 families. No histologic material was recoverable from family B. In FIG. 3, Cytoplasmic DICER1 protein staining is seen in both epithelial and mesenchymal components in 13 week gestation fetal lung and normal lung in 18 month-old child from Family X whose tumor epithelium is shown below in (D). FIGS. 3A and 3B. Six of seven PPBs with an epithelial component to the tumor showed absent staining in the surface epithelial cells (arrows) but retention of staining of the mesenchymal tumor cells (representative fields from three separate tumors from Families C, D, E shown here). See FIG. 3C, 3D, 3E. Note Family C had a missense mutation but still lacks DICER1 protein expression by immunohistochemistry. One of the seven tumors with epithelial component showed positive staining in the epithelium in the single slide available for analysis (Family G). See FIG. 3F.

Interestingly, the malignant mesenchymal tumor cells were positive for DICER1 protein in all 10 families. In contrast, lack of DICER1 expression was noted in tumor-associated epithelium in six of the seven families harboring Type I or II PPBs with an epithelial cystic component, including the PPB and two lung cysts from the family with the missense mutation (FIG. 3; Table 1). The areas of loss were focal in most cases and loss was clearly seen in areas overlying mesenchymal condensations (cambium layers) (FIG. 3A, B). The non-neoplastic lung adjacent to the tumor showed retained DICER1 expression in the alveolar and bronchial epithelium providing an important internal control. In the one family in which DICER1 protein expression was retained in the epithelium, the Type I PPBs did not show a proliferating mesenchymal component in the slides available (data not shown).

Western blot analysis was performed using an anti-DICER1 N-terminal antibody raised to a peptide from amino acid 749 to amino acid 798 (13D6, Abcam, Cambrige, MA) to determine if the truncated protein was present. Only family (B) was informative (families D, G and L have protein truncations that are more N-terminal than the epitope detected by the 13D6 antibody). As predicted by the RT-PCR analysis, the mutant truncated ˜99 KDa protein from proband B was not detectable (FIG. 3D).

Discussion

We demonstrate DICER1 germline mutations in 10 of 11 families showing predisposition to PPB. In nine families, the mutations result in premature truncation of the protein proximal to its functional RNase domain thus we view these as loss-of-function mutations. The missense mutation identified in a tenth family may also abrogate DICER1 function.

The IHC data demonstrate DICER1 protein is lost specifically in tumor associated epithelium suggesting the absence of DICER1 in the epithelium confers risk for malignant transformation in mesenchymal cells. The mesenchymal condensation comprising the cambium layer directly subjacent to the epithelium in early PPBs shows enhanced proliferation supporting a mechanism by which epithelial loss of DICER1 adversely impacts production of diffusible factors that regulate mesenchymal growth (FIG. 3A). Indeed, studies in the mouse demonstrate epithelial specific loss of Dicer1 in the developing lung alters epithelial-mesenchymal signaling resulting in a lung phenotype that mimics early PPB (Harris, K. S., et al. “Dicer function is essential for lung epithelium morphogenesis.” Proc. Natl. Acad. Sci. U.S.A 103 (2006): 2208-13). The current studies extend these prior observations in the mouse to human tumorigenesis and provide evidence that the key cell initiating tumorigenesis in hereditary PPB is not the mesenchymal cell as was long suspected, but rather the epithelial cell.

Our understanding of cancer has largely come from analyzing genetic aberrations within the malignant tumor population. Identification of DICER1 loss in the tumor associated benign epithelium described here provides evidence that the genetic abnormality that predisposes to PPB occurs in cells that do not themselves undergo transformation. Hill, et al. previously demonstrated experimentally that epithelial tumorigenesis can promote mesenchymal transformation through non-cell autonomous mechanisms in a murine prostate cancer model (Hill, R. et al., Cell 123:1001 (2005). Epithelial specific loss of retinoblastoma (Rb) family tumor suppressor function provided a mitogenic signal to the mesenchyme and induced a paracrine p53 response critical for suppressing malignant transformation. Accordingly, p53 loss in the stroma resulted in increased mesenchymal cell proliferation and tumorigenesis (Hill, R. et al., Cell 123:1001 (2005).

Our findings provide evidence for a non-cell autonomous mechanism of mesenchymal transformation secondary to loss of a DICER1-dependent suppressive function in lung epithelium. Interestingly, p53 mutations have been reported in late stage PPBs (32) suggesting that like Rb, DICER1 loss could induce a paracrine p53 response critical for suppressing mesenchymal transformation (Kusafuka et al, Pediatr. Hematol. And Oncol. 19:117 (2002)). Taken together, these studies highlight the importance of determining the cell of origin for mutations detected in human predisposition syndromes, and emphasize that genetic analysis of the malignant tumor cell population may not reveal the genetic events that predispose to malignant transformation.

DICER1 is a key component of a highly conserved regulatory pathway that functions to modulate multiple cellular processes including organogenesis and oncogenesis. Here, we identify DICER1 mutations in a hereditary tumor predisposition syndrome and provide evidence that DICER1 loss promotes malignant transformation through a non-cell autonomous mechanism. PPB is an important human model for understanding how loss of DICER1 (and the miRNAs it regulates) predisposes to oncogenesis since this tumor represents the first malignancy associated with germline DICER1 mutations. Given that hereditary PPB is associated with an increased risk for development of other more common malignancies, DICER1-dependent tumor suppressive mechanisms uncovered in PPB will likely apply to other more common cancers.

Any patents and/or publications referred to herein are hereby incorporated by reference.

The above specification, examples and data provide a complete description of the manufacture and use of the composition of the invention. Many embodiments of the invention can be made without departing from the spirit and scope of the invention.

TABLE 3
SEQ ID NO: 1

NM_177438 Homo sapiens dicer 1, ribonuclease
type III (DICER1), transcript variant 1,
mRNA. GI:29294651
MKSPALQPLSMAGLQLMTPASSPMGPFFGLPWQQEAIHDNIYTPRKYQ
VELLEAALDHNTIVCLNTGSGKTFIAVLLTKELSYQIRGDFSRNGKRT
VFLVNSANQVAQQVSAVRTHSDLKVGEYSNLEVNASWTKERWNQEFTK
HQVLIMTCYVALNVLKNGYLSLSDINLLVFDECHLAILDHPYREIMKL
CENCPSCPRILGLTASILNGKCDPEELEEKIQKLEKILKSNAETATDL
VVLDRYTSQPCEIVVDCGPFTDRSGLYERLLMELEEALNFINDCNISV
HSKERDSTLISKQILSDCRAVLVVLGPWCADKVAGMMVRELQKYIKHE
QEELHRKFLLFTDTFLRKIHALCEEHFSPASLDLKFVTPKVIKLLEIL
RKYKPYERQQFESVEWYNNRNQDNYVSWSDSEDDDEDEEIEEKEKPET
NFPSPFTNILCGIIFVERRYTAVVLNRLIKEAGKQDPELAYISSNFIT
GHGIGKNQPRNKQMEAEFRKQEEVLRKFRAHETNLLIATSIVEEGVDI
PKCNLVVRFDLPTEYRSYVQSKGRARAPISNYIMLADTDKIKSFEEDL
KTYKAIEKILRNKCSKSVDTGETDIDPVMDDDDVFPPYVLRPDDGGPR
VTINTAIGHINRYCARLPSDPFTHLAPKCRTRELPDGTFYSTLYLPIN
SPLRASIVGPPMSCVRLAERVVALICCEKLHKIGELDDHLMPVGKETV
KYEEELDLHDEEETSVPGRPGSTKRRQCYPKAIPECLRDSYPRPDQPC
YLYVIGMVLTTPLPDELNFRRRKLYPPEDTTRCFGILTAKPIPQIPHF
PVYTRSGEVTISIELKKSGFMLSLQMLELITRLHQYIFSHILRLEKPA
LEFKPTDADSAYCVLPLNVVNDSSTLDIDFKFMEDIEKSEARIGIPST
KYTKETPFVFKLEDYQDAVIIPRYRNFDQPHRFYVADVYTDLTPLSKF
PSPEYETFAEYYKTKYNLDLTNLNQPLLDVDHTSSRLNLLTPRHLNQK
GKALPLSSAEKRKAKWESLQNKQILVPELCAIHPIPASLWRKAVCLPS
ILYRLHCLLTAEELRAQTASDAGVGVRSLPADFRYPNLDFGWKKSIDS
KSFISISNSSSAENDNYCKHSTIVPENAAHQGANRTSSLENHDQMSVN
CRTLLSESPGKLHVEVSADLTAINGLSYNQNLANGSYDLANRDFCQGN
QLNYYKQEIPVQPTTSYSIQNLYSYENQPQPSDECTLLSNKYLDGNAN
KSTSDGSPVMAVMPGTTDTIQVLKGRMDSEQSPSIGYSSRTLGPNPGL
ILQALTLSNASDGFNLERLEMLGDSFLKHAITTYLFCTYPDAHEGRLS
YMRSKKVSNCNLYRLGKKKGLPSRMVVSIFDPPVNWLPPGYVVNQDKS
NTDKWEKDEMTKDCMLANGKLDEDYEEEDEEEESLMWRAPKEEADYED
DFLEYDQEHIRFIDNMLMGSGAFVKKISLSPFSTTDSAYEWKMPKKSS
LGSMPFSSDFEDFDYSSWDAMCYLDPSKAVEEDDFVVGFWNPSEENCG
VDTGKQSISYDLHTEQCIADKSIADCVEALLGCYLTSCGERAAQLFLC
SLGLKVLPVIKRTDREKALCPTRENFNSQQKNLSVSCAAASVASSRSS
VLKDSEYGCLKIPPRCMFDHPDADKTLNHLISGFENFEKKINYRFKNK
AYLLQAFTHASYHYNTITDCYQRLEFLGDAILDYLITKHLYEDPRQHS
PGVLTDLRSALVNNTIFASLAVKYDYHKYFKAVSPELFHVIDDFVQFQ
LEKNEMQGMDSELRRSEEDEEKEEDIEVPKAMGDIFESLAGAIYMDSG
MSLETVWQVYYPMMRPLIEKFSANVPRSPVRELLEMEPETAKFSPAER
TYDGKVRVTVEVVGKGKFKGVGRSYRIAKSAAARRALRSLKANQPQVP
NS

TABLE 4
SEQ ID NO: 2

NM_177438 Homo sapiens dicer 1, ribonuclease type III (DICER1),
transcript variant 1, mRNA. GI:168693430

| | |
|---|---|
| 1 | cggaggcgcg gcgcaggctg ctgcaggccc aggtgaatgg agtaacctga cagcggggac |
| 61 | gaggcgacgg cgagcgcgag gaaatggcgg cgggggcggc ggcgccgggc ggctccggga |
| 121 | ggcctgggct gtgacgcgcg cgccggagcg gggtccgatg gttctcgaag gcccgcggcg |
| 181 | ccccgtgctg cagtaagctg tgctagaaca aaaatgcaat gaaagaaaca ctggatgaat |
| 241 | gaaaagccct gctttgcaac ccctcagcat ggcaggcctg cagctcatga cccctgcttc |
| 301 | ctcaccaatg ggtcctttct ttggactgcc atggcaacaa gaagcaattc atgataacat |
| 361 | ttatacgcca agaaaatatc aggttgaact gcttgaagca gctctggatc ataataccat |
| 421 | cgtctgttta aacactggct cagggaagac atttattgca gtactactca ctaaagagct |
| 481 | gtcctatcag atcaggggag acttcagcag aaatggaaaa aggacggtgt tcttggtcaa |
| 541 | ctctgcaaac caggttgctc aacaagtgtc agctgtcaga actcattcag atctcaaggt |
| 601 | tggggaatac tcaaacctag aagtaaatgc atcttggaca aaagagagat ggaaccaaga |
| 661 | gtttactaag caccaggttc tcattatgac ttgctatgtc gccttgaatg ttttgaaaaa |
| 721 | tggttactta tcactgtcag acattaacct tttggtgttt gatgagtgtc atcttgcaat |
| 781 | cctagaccac ccctatcgag aaattatgaa gctctgtgaa aattgtccat catgtcctcg |
| 841 | cattttggga ctaactgctt ccattttaaa tgggaaatgt gatccagagg aattggaaga |
| 901 | aaagattcag aaactagaga aaattcttaa gagtaatgct gaaactgcaa ctgacctggt |
| 961 | ggtcttagac aggtatactt ctcagccatg tgagattgtg gtggattgtg gaccatttac |
| 1021 | tgacagaagt gggctttatg aaagactgct gatggaatta gaagaagcac ttaattttat |
| 1081 | caatgattgt aatatatctg tacattcaaa agaaagagat tctactttaa tttcgaaaca |
| 1141 | gatactatca gactgtcgtg ccgtattggt agttctggga ccctggtgtg cagataaagt |
| 1201 | agctggaatg atggtaagag aactacagaa atacatcaaa catgagcaag aggagctgca |
| 1261 | caggaaattt ttattgttta cagacacttt cctaaggaaa atacatgcac tatgtgaaga |
| 1321 | gcacttctca cctgcctcac ttgacctgaa atttgtaact cctaaagtaa tcaaactgct |
| 1381 | cgaaatctta cgcaaatata aaccatatga gcgacagcag tttgaaagcg ttgagtggta |
| 1441 | taataataga aatcaggata attatgtgtc atggagtgat tctgaggatg atgatgagga |
| 1501 | tgaagaaatt gaagaaaaag agaagccaga gacaaatttt ccttctcctt ttaccaacat |
| 1561 | tttgtgcgga attatttttg tggaaagaag atacacagca gttgtcttaa acagattgat |
| 1621 | aaaggaagct ggcaaacaag atccagagct ggcttatatc agtagcaatt tcataactgg |
| 1681 | acatggcatt gggaagaatc agcctcgcaa caaacagatg gaagcagaat tcagaaaaca |
| 1741 | ggaagaggta cttaggaaat ttcgagcaca tgagaccaac ctgcttattg caacaagtat |
| 1801 | tgtagaagag ggtgttgata taccaaaatg caacttggtg gttcgttttg atttgcccac |
| 1861 | agaatatcga tcctatgttc aatctaaagg aagagcaagg gcacccatct ctaattatat |
| 1921 | aatgttagcg gatacagaca aaataaaaag ttttgaagaa gaccttaaaa cctacaaagc |
| 1981 | tattgaaaag atcttgagaa acaagtgttc caagtcggtt gatactggtg agactgacat |
| 2041 | tgatcctgtc atggatgatg atgacgtttt cccaccatat gtgttgaggc ctgacgatgg |
| 2101 | tggtccacga gtcacaatca acacggccat tggacacatc aatagatact gtgctagatt |
| 2161 | accaagtgat ccgtttactc atctagctcc taaatgcaga acccgagagt tgcctgatgg |
| 2221 | tacattttat tcaactcttt atctgccaat taactcacct cttcgagcct ccattgttgg |
| 2281 | tccaccaatg agctgtgtac gattggctga aagagttgta gctctcattt gctgtgagaa |
| 2341 | actgcacaaa attggcgaac tggatgacca tttgatgcca gttgggaaag agactgttaa |
| 2401 | atatgaagag gagcttgatt tgcatgatga agaagagacc agtgttccag gaagaccagg |
| 2461 | ttccacgaaa cgaaggcagt gctacccaaa agcaattcca gagtgtttga gggatagtta |
| 2521 | tcccagacct gatcagccct gttacctgta tgtgatagga atggttttaa ctacaccttt |
| 2581 | acctgatgaa ctcaacttta gaaggcggaa gctctatcct cctgaagata ccacaagatg |
| 2641 | ctttggaata ctgacggcca aacccatacc tcagattcca cactttcctg tgtacacacg |
| 2701 | ctctggagag gttaccatat ccattgagtt gaagaagtct ggtttcatgt tgtctctaca |
| 2761 | aatgcttgag ttgattacaa gacttcacca gtatatattc tcacatattc ttcggcttga |
| 2821 | aaaacctgca ctagaattta aacctacaga cgctgattca gcatactgtg ttctacctct |
| 2881 | taatgttgtt aatgactcca gcactttgga tattgacttt aaattcatgg aagatattga |
| 2941 | gaagtctgaa gctcgcatag gcattcccag tacaaagtat acaaaagaaa caccctttgt |
| 3001 | ttttaaatta gaagattacc aagatgccgt tatcattcca agatatcgca attttgatca |
| 3061 | gcctcatcga ttttatgtag ctgatgtgta cactgatctt accccactca gtaaatttcc |
| 3121 | ttcccctgag tatgaaactt ttgcagaata ttataaaaca aagtacaacc ttgacctaac |
| 3181 | caatctcaac cagccactgc tggatgtgga ccacatatct tcaagactta atcttttgac |
| 3241 | acctcgacat ttgaatcaga aggggaaagc gcttccttta agcagtgctg agaagaggaa |
| 3301 | agccaaatgg gaaagtctgc agaataaaca gatactggtt ccagaactct gtgctataca |
| 3361 | tccaattcca gcatcactgt ggagaaaagc tgtttgtctc cccagcatac tttatcgcct |
| 3421 | tcactgcctt ttgactgcag aggagctaag agcccagact gccagcgatg ctggcgtggg |
| 3481 | agtcagatca cttcctgcgg attttagata ccctaactta gacttcgggt ggaaaaaatc |
| 3541 | tattgacagc aaatctttca tctcaatttc taactcctct tcagctgaaa atgataatta |
| 3601 | ctgtaagcac agcacaattg tccctgaaaa tgctgcacat caaggtgcta atagaacctc |
| 3661 | ctctctagaa aatcatgacc aaatgtctgt gaactgcaga acgttgctca gcgagtcccc |
| 3721 | tggtaagctc cacgttgaag tttcagcaga tcttacagea attaatggtc tttcttacaa |
| 3781 | tcaaaatctc gccaatggca gttatgattt agctaacaga gacttttgcc aaggaaatca |
| 3841 | gctaaattac tacaagcagg aaatacccgt gcaaccaact acctcatatt ccattcagaa |
| 3901 | tttatacagt tacgagaacc agccccagcc cagcgatgaa tgtactctcc tgagtaataa |
| 3961 | ataccttgat ggaaatgcta acaaatctac ctcagatgga agtcctgtga tggccgtaat |
| 4021 | gcctggtacg acagacacta ttcaagtgct caagggcagg atggattctg agcagagccc |
| 4081 | ttctattggg tactcctcaa ggactcttgg ccccaatcct ggacttattc ttcaggcttt |
| 4141 | gactctgtca aacgctagtg atggatttaa cctggagcgg cttgaaatgc ttggcgactc |
| 4201 | ctttttaaag catgccatca ccacatatct attttgcact taccctgatg cgcatgaggg |
| 4261 | ccgcctttca tatatgagaa gcaaaaaggt cagcaactgt aatctgtatc gccttggaaa |
| 4321 | aaagaaggga ctacccagcc gcatggtggt gtcaatattt gatccccctg tgaattggct |
| 4381 | tcctcctggt tatgtagtaa atcaagacaa aagcaacaca gataaatggg aaaaagatga |
| 4441 | aatgacaaaa gactgcatgc tggcgaatgg caaactggat gaggattacg aggaggagga |
| 4501 | tgaggaggag gagagcctga tgtggagggc tccgaaggaa gaggctgact atgaagatga |
| 4561 | tttcctggag tatgatcagg aacatatcag atttatagat aatatgttaa tggggtcagg |
| 4621 | agcttttgta aagaaaatct ctctttctcc tttttcaacc actgattctg catatgaatg |
| 4681 | gaaaatgccc aaaaaatcct ccttaggtag tatgccattt tcatcagatt ttgaggattt |
| 4741 | tgactacagc tcttgggatg caatgtgcta tctggatcct agcaaagctg ttgaagaaga |
| 4801 | tgactttgtg gtggggttct ggaatccatc agaagaaaac tgtggtgttg acacgggaaa |
| 4861 | gcagtccatt tcttacgact tgcacactga gcagtgtatt gctgacaaaa gcatagcgga |
| 4921 | ctgtgtggaa gccctgctgg gctgctattt aaccagctgt ggggagaggg ctgctcagct |
| 4981 | tttcctctgt tcactggggc tgaaggtgct cccggtaatt aaaaggactg atcgggaaaa |
| 5041 | ggccctgtgc cctactcggg agaatttcaa cagccaacaa aagaaccttt cagtgagctg |
| 5101 | tgctgctgct tctgtggcca gttcacgctc ttctgtattg aaagactcgg aatatggttg |
| 5161 | tttgaagatt ccaccaagat gtatgtttga tcatccagat gcagataaaa cactgaatca |
| 5221 | ccttatatcg gggtttgaaa attttgaaaa gaaaatcaac tacagattca agaataaggc |
| 5281 | ttaccttctc caggctttta cacatgcctc ctaccactac aatactatca ctgattgtta |
| 5341 | ccagcgctta gaattcctgg gagatgcgat tttggactac ctcataacca agcaccttta |
| 5401 | tgaagacccg cggcagcact ccccgggggt cctgacagac ctgcggtctg ccctggtcaa |
| 5461 | caacaccatc tttgcatcgc tggctgtaaa gtacgactac cacaagtact tcaaagctgt |
| 5521 | ctctcctgag ctcttccatg tcattgatga ctttgtgcag tttcagcttg agaagaatga |
| 5581 | aatgcaagga atggattctg agcttaggag atctgaggag gatgaagaga aagaagagga |
| 5641 | tattgaagtt ccaaaggcca tgggggatat ttttgagtcg cttgctggtg ccatttacat |
| 5701 | ggatagtggg atgtcactgg agacagtctg gcaggtgtac tatcccatga tgcggccact |
| 5761 | aatagaaaag ttttctgcaa atgtaccccg ttcccctgtg cgagaattgc ttgaaatgga |
| 5821 | accagaaact gccaaattta gcccggctga gagaacttac gacgggaagg tcagagtcac |
| 5881 | tgtggaagta gtaggaaagg ggaaatttaa aggtgttggt cgaagttaca ggattgccaa |
| 5941 | atctgcagca gcaagaagag ccctccgaag cctcaaagct aatcaacctc aggttcccaa |
| 6001 | tagctgaaac cgctttttaa aattcaaaac aagaaacaaa acaaaaaaaa ttaaggggaa |
| 6061 | aattatttaa atcggaaagg aagacttaaa gttgttagtg agtggaatga attgaaggca |
| 6121 | gaatttaaag tttggttgat aacaggatag ataacagaat aaaacattta acatatgtat |
| 6181 | aaaattttgg aactaattgt agttttagtt ttttgcgcaa acacaatctt atcttctttc |
| 6241 | ctcacttctg ctttgtttaa atcacaagag tgctttaatg atgacattta gcaagtgctc |
| 6301 | aaaataattg acaggttttg tttttttttt tttgagttta tgtcagcttt gcttagtgtt |
| 6361 | agaaggccat ggagcttaaa cctccagcag tccctaggat gatgtagatt cttctccatc |
| 6421 | tctccgtgtg tgcagtagtg ccagtcctgc agtagttgat aagctgaata gaaagataag |
| 6481 | gttttcgaga ggagaagtgc gccaatgttg tcttttcttt ccacgttata ctgtgtaagg |
| 6541 | tgatgttccc ggtcgctgtt gcacctgata gtaagggaca gatttttaat gaacattggc |
| 6601 | tggcatgttg gtgaatcaca ttttagtttt ctgatgccac atagtcttgc ataaaaaagg |
| 6661 | gttcttgcct taaaagtgaa accttcatgg atagtcttta atctctgatc tttttggaac |
| 6721 | aaactgtttt acattccttt cattttatta tgcattagac gttgagacag cgtgatactt |
| 6781 | acaactcact agtatagttg taacttatta caggatcata ctaaaatttc tgtcatatgt |
| 6841 | atactgaaga cattttaaaa accagaatat gtagtctacg gatatttttt atcataaaaa |
| 6901 | tgatctttgg ctaaacaccc cattttacta aagtcctcct gccaggtagt tcccactgat |
| 6961 | ggaaatgttt atggcaaata attttgcctt ctaggctgtt gctctaacaa aataaacctt |
| 7021 | agacatatca cacctaaaat atgctgcaga ttttataatt gattggttac ttatttaaga |
| 7081 | agcaaaacac agcaccttta cccttagtct cctcacataa atttcttact atacttttca |
| 7141 | taatgttgca tgcatatttc acctaccaaa gctgtgctgt taatgccgtg aaagtttaac |
| 7201 | gtttgcgata aactgccgta attttgatac atctgtgatt taggtcatta atttagataa |
| 7261 | actagctcat tatttccatc tttggaaaag gaaaaaaaaa aaaacttctt taggcatttg |
| 7321 | cctaagtttc tttaattaga cttgtaggca ctcttcactt aaatacctca gttcttcttt |
| 7381 | tcttttgcat gcatttttcc cctgtttggt gctatgttta tgtattatgc ttgaaatttt |
| 7441 | aatttttttt tttttgcact gtaactataa tacctcttaa tttacctttt taaaagctgt |
| 7501 | gggtcagtct tgcactccca tcaacatacc agtagaggtt tgctgcaatt tgccccgtta |
| 7561 | attatgcttg aagtttaaga aagctgagca gaggtgtctc atatttccca gcacatgatt |
| 7621 | ctgaacttga tgcttcgtgg aatgctgcat ttatatgtaa gtgacatttg aatactgtcc |
| 7681 | ttcctgcttt atctgcatca tccacccaca gagaaatgcc tctgtgcgag tgcaccgaca |
| 7741 | gaaaactgtc agctctgctt tctaaggaac cctgagtgag gggggtatta agcttctcca |
| 7801 | gtgttttttg ttgtctccaa tcttaaactt aaattgagat ctaaattatt aaacgagttt |
| 7861 | ttgagcaaat taggtgactt gttttaaaaa tatttaattc cgatttggaa ccttagatgt |
| 7921 | ctatttgatt ttttaaaaaa ccttaatgta agatatgacc agttaaaaca aagcaattct |
| 7981 | tgaattatat aactgtaaaa gtgtgcagtt aacaaggctg gatgtgaatt ttattctgag |
| 8041 | ggtgatttgt gatcaagttt aatcacaaat ctcttaatat ttataaacta cctgatgcca |
| 8101 | ggagcttagg gctttgcatt gtgtctaata cattgatccc agtgttacgg gattctcttg |
| 8161 | attcctggca ccaaaatcag attgttttca cagttatgat tcccagtggg agaaaaatgc |
| 8221 | ctcaatatat ttgtaacctt aagaagagta tttttttgtt aatactaaga tgttcaaact |
| 8281 | tagacatgat taggtcatac attctcaggg gttcaaattt ccttctacca ttcaaatgtt |
| 8341 | ttatcaacag caaacttcag ccgtttcact ttttgttgga gaaaaatagt agattttaat |
| 8401 | ttgactcaca gtttgaagca ttctgtgatc ccctggttac tgagttaaaa aataaaaaag |
| 8461 | tacgagttag acatatgaaa tggttatgaa cgcttttgtg ctgctgattt ttaatgctgt |
| 8521 | aaagttttcc tgtgtttagc ttgttgaaat gttttgcatc tgtcaattaa ggaaaaaaaa |
| 8581 | aatcactcta tgttgcccca ctttagagcc ctgtgtgcca ccctgtgttc ctgtgattgc |
| 8641 | aatgtgagac cgaatgtaat atggaaaacc taccagtggg gtgtggttgt gccctgagca |
| 8701 | cgtgtgtaaa ggactgggga ggcgtgtctt gaaaaagcaa ctgcagaaat tccttatgat |
| 8761 | gattgtgtgc aagttagtta acatgaacct tcatttgtaa attttttaaa atttctttta |
| 8821 | taatatgctt tccgcagtcc taactatgct gcgttttata atagcttttt cccttctgtt |
| 8881 | ctgttcatgt agcacagata agcattgcac ttggtaccat gctttacctc atttcaagaa |
| 8941 | aatatgctta acagagagga aaaaaatgtg gtttggcctt gctgctgttt tgatttatgg |
| 9001 | aatttgaaaa agataattat aatgcctgca atgtgtcata tactcgcaca acttaaatag |
| 9061 | gtcatttttg tctgtggcat ttttactgtt tgtgaaagta tgaaacagat ttgttaactg |
| 9121 | aactcttaat tatgttttta aaatgtttgt tatatttctt ttcttttttc ttttatatta |
| 9181 | cgtgaagtga tgaaatttag aatgacctct aacactcctg taattgtctt ttaaaatact |
| 9241 | gatattttta tttgttaata atactttgcc ctcagaaaga ttctgatacc ctgccttgac |
| 9301 | aacatgaaac ttgaggctgc tttggttcat gaatccaggt gttcccccgg cagtcggctt |
| 9361 | cttcagtcgc tccctggagg caggtgggca ctgcagagga tcactggaat ccagatcgag |
| 9421 | cgcagttcat gcacaaggcc ccgttgattt aaaatattgg atcttgctct gttagggtgt |
| 9481 | ctaatccctt tacacaagat tgaagccacc aaactgagac cttgatacct ttttttaact |
| 9541 | gcatctgaaa ttatgttaag agtctttaac ccatttgcat tatctgcaga agagaaactc |
| 9601 | atgtcatgtt tattacctat atggttgttt taattacatt tgaataatta tatttttcca |
| 9661 | accactgatt acttttcagg aatttaatta tttccagata aatttcttta ttttatattg |
| 9721 | tacatgaaaa gttttaaaga tatgtttaag accaagacta ttaaaatgat ttttaaagtt |
| 9781 | gttggagacg ccaatagcaa tatctaggaa atttgcattg agaccattgt attttccact |
| 9841 | agcagtgaaa atgatttttc acaactaact tgtaaatata ttttaatcat tacttctttt |
| 9901 | tttctagtcc atttttattt ggacatcaac cacagacaat ttaaatttta tagatgcact |
| 9961 | aagaattcac tgcagcagca ggttacatag caaaaatgca aaggtgaaca ggaagtaaat |
| 10021 | ttctggcttt tctgctgtaa atagtgaagg aaaattacta aaatcaagta aaactaatgc |
| 10081 | atattatttg attgacaata aaatatttac catcacatgc tgcagctgtt ttttaaggaa |
| 10141 | catgatgtca ttcattcata cagtaatcat gctgcagaaa tttgcagtct gcaccttatg |
| 10201 | gatcacaatt acctttagtt gttttttttg taataattgt agccaagtaa atctccaata |
| 10261 | aagttatcgt ctgttcaaaa aaaaaaaaaa aaaaaaaaaa aaaaaaaaaa aaaaaaaaaa |
| 10321 | aaa |

TABLE 5
SEQ ID NO: 3

NP_803187 dicer1 [Homo sapiens] GI:29294651

| | |
|---|---|
| 1 | mkspalqpls maglqlmtpa sspmgpffgl pwqqeaihdn iytprkyqve lleaaldhnt |
| 61 | ivclntgsgk tfiavlltke lsyqirgdfs rngkrtvflv nsanqvaqqv savrthsdlk |
| 121 | vgeysnlevn aswtkerwnq eftkhqvlim tcyvalnvlk ngylslsdin llvfdechla |
| 181 | ildhpyreim klcencpscp rilgltasil ngkcdpeele ekiqklekil ksnaetatdl |
| 241 | vvldrytsqp ceivvdcgpf tdrsglyerl lmeleealnf indcnisvhs kerdstlisk |
| 301 | qilsdcravl vvlgpwcadk vagmmvrelq kyikheqeel hrkfllftdt flrkihalce |
| 361 | ehfspasldl kfvtpkvikl leilrkykpy erqqfesvew ynnrnqdnyv swsdseddde |
| 421 | deeieekekp etnfpspftn ilcgiifver rytavvlnrl ikeagkqdpe layissnfit |
| 481 | ghgigknqpr nkqmeaefrk qeevlrkfra hetnlliats iveegvdipk cnlvvrfdlp |
| 541 | teyrsyvqsk grarapisny imladtdkik sfeedlktyk aiekilrnkc sksvdtgetd |
| 601 | idpvmddddv fppyvlrpdd ggprvtinta ighinrycar lpsdpfthla pkcrtrelpd |
| 661 | gtfystlylp insplrasiv gppmscvrla ervvalicce klhkigeldd hlmpvgketv |
| 721 | kyeeeldlhd eeetsvpgrp gstkrrqcyp kaipeclrds yprpdqpcyl yvigmvlttp |
| 781 | lpdelnfrrr klyppedttr cfgiltakpi pqiphfpvyt rsgevtisie lkksgfmlsl |
| 841 | qmlelitrlh qyifshilrl ekpalefkpt dadsaycvlp lnvvndsstl didfkfmedi |
| 901 | eksearigip stkytketpf vfkledygda viipryrnfd qphrfyvadv ytdltplskf |
| 961 | pspeyetfae yyktkynldl tnlnqplldv dhtssrlnll tprhlnqkgk alplssaekr |
| 1021 | kakweslqnk qilvpelcai hpipaslwrk avclpsilyr lhclltaeel raqtasdagv |
| 1081 | gvrslpadfr ypnldfgwkk sidsksfisi snsssaendn yckhstivpe naahqganrt |
| 1141 | sslenhdqms vncrtllses pgklhvevsa dltainglsy nqnlangsyd lanrdfcqgn |
| 1201 | qlnyykqeip vqpttsysiq nlysyenqpq psdectllsn kyldgnanks tsdgspvmav |
| 1261 | mpgttdtiqv lkgrmdseqs psigyssrtl gpnpglilqa ltlsnasdgf nlerlemlgd |
| 1321 | sflkhaitty lfctypdahe grlsymrskk vsncnlyrlg kkkglpsrmv vsifdppvnw |
| 1381 | lppgyvvnqd ksntdkwekd emtkdcmlan gkldedyeee deeeeslmwr apkeeadyed |
| 1441 | dfleydqehi rfidnmlmgs gafvkkisls pfsttdsaye wkmpkksslg smpfssdfed |
| 1501 | fdysswdamc yldpskavee ddfvvgfwnp seencgvdtg kqsisydlht eqciadksia |
| 1561 | dcveallgcy ltscgeraaq lflcslglkv lpvikrtdre kalcptrenf nsqqknlsvs |
| 1621 | caaasvassr ssvlkdseyg clkipprcmf dhpdadktln hlisgfenfe kkinyrfknk |
| 1681 | ayllqaftha syhyntitdc yqrleflgda ildylitkhl yedprqhspg vltdlrsalv |
| 1741 | nntifaslav kydyhkyfka vspelfhvid dfvqfqlekn emqgmdselr rseedeekee |
| 1801 | dievpkamgd ifeslagaiy mdsgmsletv wqvyypmmrp liekfsanvp rspvrellem |
| 1861 | epetakfspa ertydgkvrv tvevvgkgkf kgvgrsyria ksaaarralr slkanqpqvp |
| 1921 | ns |

TABLE 6
Confirmation of SNP in DICER1 SEQ ID NO: 4

>gi|168693430|ref|NM_177438.21| Homo sapiens dicer 1, ribonuclease
type III (DICER1), transcript variant 1, mRNA
CGGAGGCGCGGCGCAGGCTGCTGCAGGCCCAGGTGAATGGAGTAACCTGACAGCGGGGACGAGGCGACGG
CGAGCGCGAGGAAATGGCGGCGGGGGCGGCGGCGCCGGGCGGCTCCGGGAGGCCTGGGCTGTGACGCGCG
CGCCGGAGCGGGGTCCGATGGTTCTCGAAGGCCCGCGGCGCCCCGTGCTGCAGTAAGCTGTGCTAGAACA
AAAATGCAATGAAAGAAACACTGGATGAATGAAAAGCCCTGCTTTGCAACCCCTCAGCATGGCAGGCCTG
CAGCTCATGACCCCTGCTTCCTCACCAATGGGTCCTTTCTTTGGACTGCCATGGCAACAAGAAGCAATTC
ATGATAACATTTATACGCCAAGAAAATATCAGGTTGAACTGCTTGAAGCAGCTCTGGATCATAATACCAT
CGTCTGTTTAAACACTGGCTCAGGGAAGACATTTATTGCAGTACTACTCACTAAAGAGCTGTCCTATCAG
ATCAGGGGAGACTTCAGCAGAAATGGAAAAAGGACGGTGTTCTTGGTCAACTCTGCAAACCAGGTTGCTC
AACAAGTGTCAGCTGTCAGAACTCATTCAGATCTCAAGGTTGGGGAATACTCAAACCTAGAAGTAAATGC
ATCTTGGACAAAAGAGAGATGGAACCAAGAGTTTACTAAGCACCAGGTTCTCATTATGACTTGCTATGTC
GCCTTGAATGTTTTGAAAAATGGTTACTTATCACTGTCAGACATTAACCTTTTGGTGTTTGATGAGTGTC
ATCTTGCAATCCTAGACCACCCCTATCGAGAAATTATGAAGCTCTGTGAAAATTGTCCATCATGTCCTCG
CATTTTGGGACTAACTGCTTCCATTTTAAATGGGAAATGTGATCCAGAGGAATTGGAAGAAAAGATTCAG
AAACTAGAGAAAATTCTTAAGAGTAATGCTGAAACTGCAACTGACCTGGTGGTCTTAGACAGGTATACTT
CTCAGCCATGTGAGATTGTGGTGGATTGTGGACCATTTACTGACAGAAGTGGGCTTTATGAAAGACTGCT
GATGGAATTAGAAGAAGCACTTAATTTTATCAATGATTGTAATATATCTGTACATTCAAAAGAAAGAGAT
TCTACTTTAATTTCGAAACAGATACTATCAGACTGTCGTGCCGTATTGGTAGTTCTGGGACCCTGGTGTG
CAGATAAAGTAGCTGGAATGATGGTAAGAGAACTACAGAAATACATCAAACATGAGCAAGAGGAGCTGCA
CAGGAAATTTTTATTGTTTACAGACACTTTCCTAAGGAAAATACATGCACTATGTGAAGAGCACTTCTCA
CCTGCCTCACTTGACCTGAAATTTGTAACTCCTAAAGTAATCAAACTGCTCGAAATCTTACGCAAATATA
AACCATATGAGCGACAGCAGTTTGAAAGCGTTGAGTGGTATAATAATAGAAATCAGGATAATTATGTGTC
ATGGAGTGATTCTGAGGATGATGATGAGGATGAAGAAATTGAAGAAAAAGAGAAGCCAGAGACAAATTTT
CCTTCTCCTTTTACCAACATTTTGTGCGGAATTATTTTTGTGGAAAGAAGATACACAGCAGTTGTCTTAA
ACAGATTGATAAAGGAAGCTGGCAAACAAGATCCAGAGCTGGCTTATATCAGTAGCAATTTCATAACTGG
ACATGGCATTGGGAAGAATCAGCCTCGCAACAAACAGATGGAAGCAGAATTCAGAAAACAGGAAGAGGTA
CTTAGGAAATTTCGAGCACATGAGACCAACCTGCTTATTGCAACAAGTATTGTAGAAGAGGGTGTTGATA
TACCAAAATGCAACTTGGTGGTTCGTTTTGATTTGCCCACAGAATATCGATCCTATGTTCAATCTAAAGG
AAGAGCAAGGGCACCCATCTCTAATTATATAATGTTAGCGGATACAGACAAAATAAAAAGTTTTGAAGAA
GACCTTAAAACCTACAAAGCTATTGAAAAGATCTTGAGAAACAAGTGTTCCAAGTCGGTTGATACTGGTG
AGACTGACATTGATCCTGTCATGGATGATGATGACGTTTTCCCACCATATGTGTTGAGGCCTGACGATGG
TGGTCCACGAGTCACAATCAACACGGCCATTGGACACATCAATAGATACTGTGCTAGATTACCAAGTGAT
CCGTTTACTCATCTAGCTCCTAAATGCAGAACCCGAGAGTTGCCTGATGGTACATTTTATTCAACTCTTT
ATCTGCCAATTAACTCACCTCTTCGAGCCTCCATTGTTGGTCCACCAATGAGCTGTGTACGATTGGCTGA
AAGAGTTGTAGCTCTCATTTGCTGTGAGAAACTGCACAAAATTGGCGAACTGGATGACCATTTGATGCCA
GTTGGGAAAGAGACTGTTAAATATGAAGAGGAGCTTGATTTGCATGATGAAGAAGAGACCAGTGTTCCAG
GAAGACCAGGTTCCACGAAACGAAGGCAGTGCTACCCAAAAGCAATTCCAGAGTGTTTGAGGGATAGTTA
TCCCAGACCTGATCAGCCCTGTTACCTGTATGTGATAGGAATGGTTTTAACTACACCTTTACCTGATGAA
CTCAACTTTAGAAGGCGGAAGCTCTATCCTCCTGAAGATACCACAAGATGCTTTGGAATACTGACGGCCA
AACCCATACCTCAGATTCCACACTTTCCTGTGTACACACGCTCTGGAGAGGTTACCATATCCATTGAGTT
GAAGAAGTCTGGTTTCATGTTGTCTCTACAAATGCTTGAGTTGATTACAAGACTTCACCAGTATATATTC
TCACATATTCTTCGGCTTGAAAAACCTGCACTAGAATTTAAACCTACAGACGCTGATTCAGCATACTGTG
TTCTACCTCTTAATGTTGTTAATGACTCCAGCACTTTGGATATTGACTTTAAATTCATGGAAGATATTGA
GAAGTCTGAAGCTCGCATAGGCATTCCCAGTACAAAGTATACAAAAGAAACACCCTTTGTTTTTAAATTA
GAAGATTACCAAGATGCCGTTATCATTCCAAGATATCGCAATTTTGATCAGCCTCATCGATTTTATGTAG
CTGATGTGTACACTGATCTTACCCCACTCAGTAAATTTCCTTCCCCTGAGTATGAAACTTTTGCAGAATA
TTATAAAACAAAGTACAACCTTGACCTAACCAATCTCAACCAGCCACTGCTGGATGTGGACCACACATCT
TCAAGACTTAATCTTTTGACACCTCGACATTTGAATCAGAAGGGGAAAGCGCTTCCTTTAAGCAGTGCTG
AGAAGAGGAAAGCCAAATGGGAAAGTCTGCAGAATAAACAGATACTGGTTCCAGAACTCTGTGCTATACA
TCCAATTCCAGCATCACTGTGGAGAAAAGCTGTTTGTCTCCCCAGCATACTTTATCGCCTTCACTGCCTT
TTGACTGCAGAGGAGCTAAGAGCCCAGACTGCCAGCGATGCTGGCGTGGGAGTCAGATCACTTCCTGCGG
ATTTTAGATACCCTAACTTAGACTTCGGGTGGAAAAAATCTATTGACAGCAAATCTTTCATCTCAATTTC
TAACTCCTCTTCAGCTGAAAATGATAATTACTGTAAGCACAGCACAATTGTCCCTGAAAATGCTGCACAT
CAAGGTGCTAATAGAACCTCCTCTCTAGAAAATCATGACCAAATGTCTGTGAACTGCAGAACGTTGCTCA
GCGAGTCCCCTGGTAAGCTCCACGTTGAAGTTTCAGCAGATCTTACAGCAATTAATGGTCTTTCTTACAA
TCAAAATCTCGCCAATGGCAGTTATGATTTAGCTAACAGAGACTTTTGCCAAGGAAATCAGCTAAATTAC
TACAAGCAGGAAATACCCGTGCAACCAACTACCTCATATTCCATTCAGAATTTATACAGTTACGAGAACC
AGCCCCAGCCCAGCGATGAATGTACTCTCCTGAGTAATAAATACCTTGATGGAAATGCTAACAAATCTAC
CTCAGATGGAAGTCCTGTGATGGCCGTAATGCCTGGTACGACAGACACTATTCAAGTGCTCAAGGGCAGG
ATGGATTCTGAGCAGAGCCCTTCTATTGGGTACTCCTCAAGGACTCTTGGCCCCAATCCTGGACTTATTC
TTCAGGCTTTGACTCTGTCAAACGCTAGTGATGGATTTAACCTGGAGCGGCTTGAAATGCTTGGCGACTC
CTTTTTAAAGCATGCCATCACCACATATCTATTTTGCACTTACCCTGATGCGCATGAGGGCCGCCTTTCA
TATATGAGAAGCAAAAAGGTCAGCAACTGTAATCTGTATCGCCTTGGAAAAAAGAAGGGACTACCCAGCC
GCATGGTGGTGTCAATATTTGATCCCCCTGTGAATTGGCTTCCTCCTGGTTATGTAGTAAATCAAGACAA
AAGCAACACAGATAAATGGGAAAAAGATGAAATGACAAAAGACTGCATGCTGGCGAATGGCAAACTGGAT
GAGGATTACGAGGAGGAGGATGAGGAGGAGGAGAGCCTGATGTGGAGGGCTCCGAAGGAAGAGGCTGACT
ATGAAGATGATTTCCTGGAGTATGATCAGGAACATATCAGATTTATAGATAATATGTTAATGGGGTCAGG
AGCTTTTGTAAAGAAAATCTCTCTTTCTCCTTTTTCAACCACTGATTCTGCATATGAATGGAAAATGCCC
AAAAAATCCTCCTTAGGTAGTATGCCATTTTCATCAGATTTTGAGGATTTTGACTACAGCTCTTGGGATG
CAATGTGCTATCTGGATCCTAGCAAAGCTGTTGAAGAAGATGACTTTGTGGTGGGGTTCTGGAATCCATC
AGAAGAAAACTGTGGTGTTGACACGGGAAAGCAGTCCATTTCTTACGACTTGCACACTGAGCAGTGTATT
GCTGACAAAAGCATAGCGGACTGTGTGGAAGCCCTGCTGGGCTGCTATTTAACCAGCTGTGGGGAGAGGG
CTGCTCAGCTTTTCCTCTGTTCACTGGGGCTGAAGGTGCTCCCGGTAATTAAAAGGACTGATCGGGAAAA
GGCCCTGTGCCCTACTCGGGAGAATTTCAACAGCCAACAAAAGAACCTTTCAGTGAGCTGTGCTGCTGCT
TCTGTGGCCAGTTCACGCTCTTCTGTATTGAAAGACTCGGAATATGGTTGTTTGAAGATTCCACCAAGAT
GTATGTTTGATCATCCAGATGCAGATAAAACACTGAATCACCTTATATCGGGGTTTGAAAATTTTGAAAA
GAAAATCAACTACAGATTCAAGAATAAGGCTTACCTTCTCCAGGCTTTTACACATGCCTCCTACCACTAC
AATACTATCACTGATTGTTACCAGCGCTTAGAATTCCTGGGAGATGCGATTTTGGACTACCTCATAACCA
AGCACCTTTATGAAGACCCGCGGCAGCACTCCCCGGGGGTCCTGACAGACCTGCGGTCTGCCCTGGTCAA
CAACACCATCTTTGCATCGCTGGCTGTAAAGTACGACTACCACAAGTACTTCAAAGCTGTCTCTCCTGAG
CTCTTCCATGTCATTGATGACTTTGTGCAGTTTCAGCTTGAGAAGAATGAAATGCAAGGAATGGATTCTG
AGCTTAGGAGATCTGAGGAGGATGAAGAGAAAGAAGAGGATATTGAAGTTCCAAAGGCCATGGGGGATAT
TTTTGAGTCGCTTGCTGGTGCCATTTACATGGATAGTGGGATGTCACTGGAGACAGTCTGGCAGGTGTAC
TATCCCATGATGCGGCCACTAATAGAAAAGTTTTCTGCAAATGTACCCCGTTCCCCTGTGCGAGAATTGC
TTGAAATGGAACCAGAAACTGCCAAATTTAGCCCGGCTGAGAGAACTTACGACGGGAAGGTCAGAGTCAC
TGTGGAAGTAGTAGGAAAGGGGAAATTTAAAGGTGTTGGTCGAAGTTACAGGATTGCCAAATCTGCAGCA
GCAAGAAGAGCCCTCCGAAGCCTCAAAGCTAATCAACCTCAGGTTCCCAATAGCTGAAACCGCTTTTTAA
AATTCAAAACAAGAAACAAAACAAAAAAAATTAAGGGGAAAATTATTTAAATCGGAAAGGAAGACTTAAA
GTTGTTAGTGAGTGGAATGAATTGAAGGCAGAATTTAAAGTTTGGTTGATAACAGGATAGATAACAGAAT
AAAACATTTAACATATGTATAAAATTTTGGAACTAATTGTAGTTTTAGTTTTTTGCGCAAACACAATCTT
ATCTTCTTTCCTCACTTCTGCTTTGTTTAAATCACAAGAGTGCTTTAATGATGACATTTAGCAAGTGCTC
AAAATAATTGACAGGTTTTGTTTTTTTTTTTTTGAGTTTATGTCAGCTTTGCTTAGTGTTAGAAGGCCAT
GGAGCTTAAACCTCCAGCAGTCCCTAGGATGATGTAGATTCTTCTCCATCTCTCCGTGTGTGCAGTAGTG
CCAGTCCTGCAGTAGTTGATAAGCTGAATAGAAAGATAAGGTTTTCGAGAGGAGAAGTGCGCCAATGTTG
TCTTTTCTTTCCACGTTATACTGTGTAAGGTGATGTTCCCGGTCGCTGTTGCACCTGATAGTAAGGGACA
GATTTTTAATGAACATTGGCTGGCATGTTGGTGAATCACATTTTAGTTTTCTGATGCCACATAGTCTTGC
ATAAAAAAGGGTTCTTGCCTTAAAAGTGAAACCTTCATGGATAGTCTTTAATCTCTGATCTTTTTGGAAC
AAACTGTTTTACATTCCTTTCATTTTATTATGCATTAGACGTTGAGACAGCGTGATACTTACAACTCACT
AGTATAGTTGTAACTTATTACAGGATCATACTAAAATTTCTGTCATATGTATACTGAAGACATTTTAAAA
ACCAGAATATGTAGTCTACGGATATTTTTTATCATAAAAATGATCTTTGGCTAAACACCCCATTTTACTA
AAGTCCTCCTGCCAGGTAGTTCCCACTGATGGAAATGTTTATGGCAAATAATTTTGCCTTCTAGGCTGTT
GCTCTAACAAAATAAACCTTAGACATATCACACCTAAAATATGCTGCAGATTTTATAATTGATTGGTTAC
TTATTTAAGAAGCAAAACACAGCACCTTTACCCTTAGTCTCCTCACATAAATTTCTTACTATACTTTTCA
TAATGTTGCATGCATATTTCACCTACCAAAGCTGTGCTGTTAATGCCGTGAAAGTTTAACGTTTGCGATA
AACTGCCGTAATTTTGATACATCTGTGATTTAGGTCATTAATTTAGATAAACTAGCTCATTATTTCCATC
TTTGGAAAAGGAAAAAAAAAAAAACTTCTTTAGGCATTTGCCTAAGTTTCTTTAATTAGACTTGTAGGCA
CTCTTCACTTAAATACCTCAGTTCTTCTTTTCTTTTGCATGCATTTTTCCCCTGTTTGGTGCTATGTTTA
TGTATTATGCTTGAAATTTTAATTTTTTTTTTTTTGCACTGTAACTATAATACCTCTTAATTTACCTTTT
TAAAAGCTGTGGGTCAGTCTTGCACTCCCATCAACATACCAGTAGAGGTTTGCTGCAATTTGCCCCGTTA
ATTATGCTTGAAGTTTAAGAAAGCTGAGCAGAGGTGTCTCATATTTCCCAGCACATGATTCTGAACTTGA
TGCTTCGTGGAATGCTGCATTTATATGTAAGTGACATTTGAATACTGTCCTTCCTGCTTTATCTGCATCA
TCCACCCACAGAGAAATGCCTCTGTGCGAGTGCACCGACAGAAAACTGTCAGCTCTGCTTTCTAAGGAAC
CCTGAGTGAGGGGGGTATTAAGCTTCTCCAGTGTTTTTTGTTGTCTCCAATCTTAAACTTAAATTGAGAT
CTAAATTATTAAACGAGTTTTTGAGCAAATTAGGTGACTTGTTTTAAAAATATTTAATTCCGATTTGGAA
CCTTAGATGTCTATTTGATTTTTTAAAAAACCTTAATGTAAGATATGACCAGTTAAAACAAAGCAATTCT
TGAATTATATAACTGTAAAAGTGTGCAGTTAACAAGGCTGGATGTGAATTTTATTCTGAGGGTGATTTGT
GATCAAGTTTAATCACAAATCTCTTAATATTTATAAACTACCTGATGCCAGGAGCTTAGGGCTTTGCATT
GTGTCTAATACATTGATCCCAGTGTTACGGGATTCTCTTGATTCCTGGCACCAAAATCAGATTGTTTTCA
CAGTTATGATTCCCAGTGGGAGAAAAATGCCTCAATATATTTGTAACCTTAAGAAGAGTATTTTTTTGTT
AATACTAAGATGTTCAAACTTAGACATGATTAGGTCATACATTCTCAGGGGTTCAAATTTCCTTCTACCA
TTCAAATGTTTTATCAACAGCAAACTTCAGCCGTTTCACTTTTTGTTGGAGAAAAATAGTAGATTTTAAT
TTGACTCACAGTTTGAAGCATTCTGTGATCCCCTGGTTACTGAGTTAAAAAATAAAAAAGTACGAGTTAG
ACATATGAAATGGTTATGAACGCTTTTGTGCTGCTGATTTTTAATGCTGTAAAGTTTTCCTGTGTTTAGC
TTGTTGAAATGTTTTGCATCTGTCAATTAAGGAAAAAAAAAATCACTCTATGTTGCCCCACTTTAGAGCC
CTGTGTGCCACCCTGTGTTCCTGTGATTGCAATGTGAGACCGAATGTAATATGGAAAACCTACCAGTGGG
GTGTGGTTGTGCCCTGAGCACGTGTGTAAAGGACTGGGGAGGCGTGTCTTGAAAAAGCAACTGCAGAAAT
TCCTTATGATGATTGTGTGCAAGTTAGTTAACATGAACCTTCATTTGTAAATTTTTTAAAATTTCTTTTA
TAATATGCTTTCCGCAGTCCTAACTATGCTGCGTTTTATAATAGCTTTTTCCCTTCTGTTCTGTTCATGT
AGCACAGATAAGCATTGCACTTGGTACCATGCTTTACCTCATTTCAAGAAAATATGCTTAACAGAGAGGA
AAAAAATGTGGTTTGGCCTTGCTGCTGTTTTGATTTATGGAATTTGAAAAAGATAATTATAATGCCTGCA
ATGTGTCATATACTCGCACAACTTAAATAGGTCATTTTTGTCTGTGGCATTTTTACTGTTTGTGAAAGTA
TGAAACAGATTTGTTAACTGAACTCTTAATTATGTTTTTAAAATGTTTGTTATATTTCTTTTCTTTTTTC
TTTTATATTACGTGAAGTGATGAAATTTAGAATGACCTCTAACACTCCTGTAATTGTCTTTTAAAATACT
GATATTTTTATTTGTTAATAATACTTTGCCCTCAGAAAGATTCTGATACCCTGCCTTGACAACATGAAAC
TTGAGGCTGCTTTGGTTCATGAATCCAGGTGTTCCCCCGGCAGTCGGCTTCTTCAGTCGCTCCCTGGAGG
CAGGTGGGCACTGCAGAGGATCACTGGAATCCAGATCGAGCGCAGTTCATGCACAAGGCCCCGTTGATTT
AAAATATTGGATCTTGCTCTGTTAGGGTGTCTAATCCCTTTACACAAGATTGAAGCCACCAAACTGAGAC
CTTGATACCTTTTTTTAACTGCATCTGAAATTATGTTAAGAGTCTTTAACCCATTTGCATTATCTGCAGA
AGAGAAACTCATGTCATGTTTATTACCTATATGGTTGTTTTAATTACATTTGAATAATTATATTTTTCCA
ACCACTGATTACTTTTCAGGAATTTAATTATTTCCAGATAAATTTCTTTATTTTATATTGTACATGAAAA
GTTTTAAAGATATGTTTAAGACCAAGACTATTAAAATGATTTTTAAAGTTGTTGGAGACGCCAATAGCAA
TATCTAGGAAATTTGCATTGAGACCATTGTATTTTCCACTAGCAGTGAAAATGATTTTTCACAACTAACT
TGTAAATATATTTTAATCATTACTTCTTTTTTTCTAGTCCATTTTTATTTGGACATCAACCACAGACAAT
TTAAATTTTATAGATGCACTAAGAATTCACTGCAGCAGCAGGTTACATAGCAAAAATGCAAAGGTGAACA
GGAAGTAAATTTCTGGCTTTTCTGCTGTAAATAGTGAAGGAAAATTACTAAAATCAAGTAAAACTAATGC
ATATTATTTGATTGACAATAAAATATTTACCATCACATGCTGCAGCTGTTTTTTAAGGAACATGATGTCA
TTCATTCATACAGTAATCATGCTGCAGAAATTTGCAGTCTGCACCTTATGGATCACAATTACCTTTAGTT
GTTTTTTTTGTAATAATTGTAGCCAAGTAAATCTCCAATAAAGTTATCGTCTGTTCAAAAAAAAAAAAAA
AAAAAAAAAAAAAAAAAAAAAAAAAAAAAAAAA

TABLE 7
SEQ ID NO: 5

CDS amino acid translation refseq
MKSPALQPLSMAGLQLMTPASSPMGPFFGLPWQQEAIHDNIYTPRKYQVELLEAALDHNTIVCLNTGSGKTFIAVLL
TKELSYQIRGDFSRNGKRTVFLVNSANQVAQQVSAVRTHSDLKVGEYSNLEVNASWTKERWNQEFTKHQVLIMTCYV
ALNVLKNGYLSLSDINLLVFDECHLAILDHPYREIMKLCENCPSCPRILGLTASILNGKCDPEELEEKIQKLEKILK
SNAETATDLVVLDRYTSQPCEIVVDCGPFTDRSGLYERLLMELEEALNFINDCNISVHSKERDSTLISKQILSDCRA
VLVVLGPWCADKVAGMMVRELQKYIKHEQEELHRKFLLFTDTFLRKIHALCEEHFSPASLDLKFVTPKVIKLLEILR
KYKPYERQQFESVEWYNNRNQDNYVSWSDSEDDDEDEEIEEKEKPETNFPSPFTNILCGIIFVERRYTAVVLNRLIK
EAGKQDPELAYISSNFITGHGIGKNQPRNKQMEAEFRKQEEVLRKFRAHETNLLIATSIVEEGVDIPKCNLVVRFDL
PTEYRSYVQSKGRARAPISNYIMLADTDKIKSFEEDLKTYKAIEKILRNKCSKSVDTGETDIDPVMDDDDVFPPYVL
RPDDGGPRVTINTAIGHINRYCARLPSDPFTHLAPKCRTRELPDGTFYSTLYLPINSPLRASIVGPPMSCVRLAERV
VALICCEKLHKIGELDDHLMPVGKETVKYEEELDLHDEEETSVPGRPGSTKRRQCYPKAIPECLRDSYPRPDQPCYL
YVIGMVLTTPLPDELNFRRRKLYPPEDTTRCFGILTAKPIPQIPHFPVYTRSGEVTISIELKKSGFMLSLQMLELIT
RLHQYIFSHILRLEKPALEFKPTDADSAYCVLPLNVVNDSSTLDIDFKFMEDIEKSEARIGIPSTKYTKETPFVFKL
EDYQDAVIIPRYRNFDQPHRFYVADVYTDLTPLSKFPSPEYETFAEYYKTKYNLDLTNLNQPLLDVDHTSSRLNLLT
PRHLNQKGKALPLSSAEKRKAKWESLQNKQILVPELCAIHPIPASLWRKAVCLPSILYRLHCLLTAEELRAQTASDA
GVGVRSLPADFRYPNLDFGWKKSIDSKSFISISNSSSAENDNYCKHSTIVPENAAHQGANRTSSLENHDQMSVNCRT
LLSESPGKLHVEVSADLTAINGLSYNQNLANGSYDLANRDFCQGNQLNYYKQEIPVQPTTSYSIQNLYSYENQPQPS
DECTLLSNKYLDGNANKSTSDGSPVMAVMPGTTDTIQVLKGRMDSEQSPSIGYSSRTLGPNPGLILQALTLSNASDG
FNLERLEMLGDSFLKHAITTYLFCTYPDAHEGRLSYMRSKKVSNCNLYRLGKKKGLPSRMVVSIFDPPVNWLPPGYV
VNQDKSNTDKWEKDEMTKDCMLANGKLDEDYEEEDEEEESLMWRAPKEEADYEDDFLEYDQEHIRFIDNMLMGSGAF
VKKISLSPFSTTDSAYEWKMPKKSSLGSMPFSSDFEDFDYSSWDAMCYLDPSKAVEEDDFVVGFWNPSEENCGVDTG
KQSISYDLHTEQCIADKSIADCVEALLGCYLTSCGERAAQLFLCSLGLKVLPVIKRTDREKALCPTRENFNSQQKNL
SVSCAAASVASSRSSVLKDSEYGCLKIPPRCMFDHPDADKTLNHLISGFENFEKKINYRFKNKAYLLQAFTHASYHY
NTITDCYQRLEFLGDAILDYLITKHLYEDPRQHSPGVLTDLRSALVNNTIFASLAVKYDYHKYFKAVSPELFHVIDD
FVQFQLEKNEMQGMDSELRRSEEDEEKEEDIEVPKAMGDIFESLAGAIYMDSGMSLETVWQVYYPMMRPLIEKFSAN
VPRSPVRELLEMEPETAKFSPAERTYDGKVRVTVEVVGKGKFKGVGRSYRIAKSAAARRALRSLKANQPQVPNS

TABLE 8
Family A
ex18 C→T
SEQ ID NO: 6 [symbol]
gattttatgtagctgatgtgtacactgatcttaccc
Family B
SEQ ID NO: 7
Aaggcggaagctctatcctcctgaagata\hat{ }ins here
Family C
Ex23 T→G
SEQ ID NO: 8
Tctgttcactggggctgaaggtgctcccggtaattaaaa
Family D
SEQ ID NO: 9
Cagatggaagcagaattcagaaaacaggaag
Family E
SEQ ID NO: 10
Actgtgctagattaccaagtgatccgtttact
Family F
SEQ ID NO: 11
ATgttagcggatacagacaaaataaaaa
Family G
SEQ ID NO: 12
Gttccacgaaacgaaggcagtgctacc\hat{ }insert
Family H
SEQ ID NO: 13
Atcttacagcaattaatggtctttcttac
Family I
SEQ ID NO: 14
Ttcgttttgatttgcccacagaatatc
Family L
SEQ ID NO: 15
Ggaagaccaggttccacgaaacgaaggcagtgctac

1. An isolated nucleic acid that comprises a first nucleic acid that encodes a portion of a DICER1 polypeptide or that comprises a portion of the DICER1 gene, wherein the first nucleic acid comprises a mutation in the nucleic acid sequence as compared to a corresponding sequence in a reference sequence having the sequence of SEQ ID NO:2, wherein the mutation in the first nucleic acid sequence decreases a function of DICER1 polypeptide.

2. An isolated nucleic acid that specifically hybridizes to the isolated nucleic acid of claim 1, wherein the nucleic acid preferentially hybridizes to the first nucleic acid sequence as compared to the reference sequence.

3. The isolated nucleic acid of claim 1, wherein the reference sequence comprises a portion of the nucleic acid sequence having the sequence of SEQ ID NO:2.

4-5. (canceled)

6. The isolated nucleic acid of claim 2, wherein the nucleic acid is a primer or a probe.

7. The isolated nucleic acid of claim 1, wherein the mutation in the first nucleic acid sequence is an amino acid substitution mutation, a missense mutation, a frameshift mutation, a deletion, an insertion, or a stop codon.

8. The isolated nucleic acid of claim 7, wherein the mutation is present in the genomic nucleic acid sequence for DICER1, and the DICER1 polypeptide lacks at least one of a ribonuclease domain.

9. The isolated nucleic acid of claim 1, wherein the mutation is located in an exon.

10. The isolated nucleic acid of claim 1, wherein the mutation is located in an intron.

11-12. (canceled)

13. The isolated nucleic acid of claim 1, wherein the mutation is located in an exon selected from the group consisting of exon 9, exon 10, exon 12, exon 14, exon 15, exon 18, exon 21, exon 23 and combinations thereof.

14. The isolated nucleic acid of claim 13, wherein the mutation is any one of the mutations shown in Table 1.

15. The isolated nucleic acid of claim 13, wherein the nucleic acid is a probe.

16. The isolated nucleic acid of claim 13, wherein the nucleic acid is a primer.

17. The isolated nucleic acid of claim 2, further comprising a detectable label.

18. The isolated nucleic acid of claim 17, wherein the detectable label is selected from the group consisting of Texas-Red®, fluorescein isothiocyanate, FAM, TAMRA, Alexa flour, a cyanine dye, a quencher, and biotin.

19. The isolated nucleic acid of claim 16, wherein the primer comprises a sequence selected from any one of the primers having the sequence of SEQ ID NOs:16 to SEQ ID NO:80.

20. A method of detecting the presence of a mutation in a DICER1 nucleic acid sequence, comprising: isolating the nucleic acid of claim 1 and sequencing the nucleic acid to determine the presence of the mutation in the first nucleic acid sequence as compared to the reference sequence having a sequence of SEQ ID NO:2.

21. A method of detecting the presence of a mutation in a DICER1 nucleic acid sequence from a subject, comprising: amplifying a nucleic acid sample from the subject with a set of primers, wherein the primers amplify at least a portion of the reference nucleic acid having the sequence of SEQ ID NO:2 that contains the location of a mutation in a nucleic acid sequence comprising a portion of the DICER1 gene, wherein the mutation in the nucleotide sequence decreases a function of DICER1 polypeptide; and determining whether the mutation is present in the amplified sample.

22-24. (canceled)

25. The method of claim 21 further comprising sequencing the amplified nucleic acid.

26. A method of determining the diagnosis or prognosis of a cancer in a subject comprising: detecting the presence or absence of a mutation in a nucleic acid having the nucleotide sequence of SEQ ID NO:2 in a sample from the subject, wherein the mutation in the nucleic acid decreases a function of DICER1 polypeptide, and wherein the presence of the mutation is indicative of the diagnosis or prognosis of the cancer in the subject.

27. The method of claim 26, wherein the cancer is selected from the group consisting of PBB, cystic nephroma, renal cysts, thyroid carcinoma, intestinal polyps, leukemia, ovarian germ cell tumors, testicular germ cell tumors, ovarian dysgerminoma, testicular seminoma, hepatic hamartomas, nasal chondromesenchymal hamartoma, Wilms tumor, rhabdomyosarcoma, synovial sarcoma, Sertoli-Leydig tumors, medulloblastoma, glioblastoma multiforme, primary brain sarcoma, ependymoma, neuroblastoma, and neurofibromatosis Type I.

28. A kit comprising a nucleic acid selected from the group consisting of a primer that amplifies a portion of a reference nucleic acid having the sequence of SEQ ID NO:2, wherein the portion of the reference nucleic acid contains the location of a mutation in the DICER1 gene, wherein the mutation in the nucleic acid sequence decreases a function of DICER1 polypeptide, a probe that hybridizes to a portion of the reference nucleic acid at the location of the mutation in the DICER1 gene, and combinations thereof.

29. The kit of claim 28, further comprising reagents for conducting an amplification reaction.

30. A kit of claim 28 wherein the probe is attached to a solid substrate.

31-33. (canceled)
